SEQUENCE LISTING (15 sequences)

SEQ ID NO: 1 - DNA, 20 bp, synthetic construct
  misc_feature 1..20: note = Synthetic: Mm.Cas9.APOE.1-A
  source 1..20: mol_type = other DNA
gaggatctacgcaaccgact

SEQ ID NO: 2 - DNA, 20 bp, synthetic construct
  misc_feature 1..20: note = Synthetic: Mm.Cas9.APOE.1-B
  source 1..20: mol_type = other DNA
caacgaggtgcacaccatgc

SEQ ID NO: 3 - DNA, 20 bp, synthetic construct
  misc_feature 1..20: note = Synthetic: Mm.Cas9.APOE.1-C
  source 1..20: mol_type = other DNA
gaggtgacagatcagctcga

SEQ ID NO: 4 - DNA, 20 bp, synthetic construct
  misc_feature 1..20: note = Synthetic: Mm.Cas9.APOE.1-D
  source 1..20: mol_type = other DNA
gacgctgtctgaccaggtcc

SEQ ID NO: 5 - DNA, 20 bp, synthetic construct
  misc_feature 1..20: note = Synthetic: Mm.Cas9.APOE.1-E
  source 1..20: mol_type = other DNA
ggacactatgacggaagtaa

SEQ ID NO: 6 - DNA, 20 bp, synthetic construct
  misc_feature 1..20: note = Synthetic: Mm.Cas9.APOE.1-F
  source 1..20: mol_type = other DNA
ctggtggagcaaggtcgcca

SEQ ID NO: 7 - DNA, 20 bp, synthetic construct
  misc_feature 1..20: note = Synthetic: Mm.Cas9.APOE.1-F
  source 1..20: mol_type = other DNA
ctggtggagcaaggtcgcca

SEQ ID NO: 8 - DNA, 53 bp, synthetic construct
  misc_feature 1..53: note = Synthetic: NGS For 1
  source 1..53: mol_type = other DNA
acactctttccctacacgacgctcttccgatctagacccaaaaagactgtagg

SEQ ID NO: 9 - DNA, 54 bp, synthetic construct
  misc_feature 1..54: note = Synthetic: NGS Rev 1
  source 1..54: mol_type = other DNA
gtgactggagttcagacgtgtgctcttccgatcttgccgagggtgaaagagctg

SEQ ID NO: 10 - DNA, 53 bp, synthetic construct
  misc_feature 1..53: note = Synthetic: NGS For 2
  source 1..53: mol_type = other DNA
acactctttccctacacgacgctcttccgatctgccttcatctccttcctgtg

SEQ ID NO: 11 - DNA, 54 bp, synthetic construct
  misc_feature 1..54: note = Synthetic: NGS Rev 2
  source 1..54: mol_type = other DNA
gtgactggagttcagacgtgtgctcttccgatctcctctgtgctctggcccagc

SEQ ID NO: 12 - DNA, 53 bp, synthetic construct
  misc_feature 1..53: note = Synthetic: NGS For 3
  source 1..53: mol_type = other DNA
acactctttccctacacgacgctcttccgatctaggctgggcaaagaggtgca

SEQ ID NO: 13 - DNA, 54 bp, synthetic construct
  misc_feature 1..54: note = Synthetic: NGS Rev 3
  source 1..54: mol_type = other DNA
gtgactggagttcagacgtgtgctcttccgatctcgcttctgcagatcctcggc

SEQ ID NO: 14 - DNA, 53 bp, synthetic construct
  misc_feature 1..53: note = Synthetic: NGS For 4
  source 1..53: mol_type = other DNA
acactctttccctacacgacgctcttccgatcttgccgaggatctgcagaagc

SEQ ID NO: 15 - DNA, 54 bp, synthetic construct
  misc_feature 1..54: note = Synthetic: NGS Rev 4
  source 1..54: mol_type = other DNA
gtgactggagttcagacgtgtgctcttccgatctgccgccctcggatgcggtca

(19) United States
(12) Patent Application Publication
(10) Pub. No.: US 2025/0011404 A1
(43) Pub. Date: Jan. 9, 2025

(54) TARGETING APOE ENHANCES T-CELL KILLING IN CANCER IMMUNOTHERAPY

(71) Applicant: Children's National Medical Center, Washington, DC (US)

(72) Inventors: Anthony Sandler, Bethesda, MD (US); Xiaofang Wu, Rockville, MD (US); Priya Srinivasan, Silver Spring, MD (US); Mousumi Basu, Rockville, MD (US)

(21) Appl. No.: 18/707,822

(22) PCT Filed: Nov. 8, 2022
(86) PCT No.: PCT/US2022/079431

§ 371 Date: May 6, 2024

Related U.S. Application Data
(60) Provisional application No. 63/277,274, filed on Nov. 9, 2021.

(51) Int. Cl. C07K 16/18; A61K 39/00; A61K 39/395; A61K 45/06; A61P 35/00; C07K 14/705; C12N 15/113

(52) U.S. Cl. CPC C07K 16/18; A61K 39/3955; A61K 39/4611; A61K 39/4631; A61K 45/06; A61P 35/00; C07K 14/705; C07K 14/70596; C12N 15/113; C07K 2317/569; C07K 2317/622; C12N 2310/14; C12N 2310/141

(57) ABSTRACT

The present disclosure provides methods for treating an apoE-expressing cancer, e.g., melanoma, by inhibiting expression or activity of apoE. In certain aspects, the methods prevent immunosuppression induced by apoE secreted by cancer cells, and enhance anti-tumor response by endogenous or adoptively transferred immune cells. The methods provided herein can be used with other therapy, including immunotherapy, such as immunotherapy using checkpoint inhibitors.

### REFERENCE TO RELATED APPLICATIONS

The present application claims the benefit of U.S. Provisional App. No. 63/277,274, filed Nov. 9, 2021, which is hereby incorporated by reference in its entirety.

### REFERENCE TO SEQUENCE LISTING

The instant application contains a Sequence Listing which has been submitted electronically in XML format and is hereby incorporated by reference in its entirety. Said XML copy, created on Nov. 7, 2022, is named 135372-02020.xml and is 20,139 bytes in size.

### BACKGROUND OF THE INVENTION

Currently, immune checkpoint blockade therapy is successfully being used for treatment of advanced non-small cell lung cancers (NSCLC), metastatic melanoma, advanced renal cell carcinoma (RCC) and metastatic squamous cell carcinoma of the head and neck (SCCHN). A few such drugs, developed and manufactured by different companies, have shown great promise in different cancer types and have been FDA approved, including antibodies to the immune checkpoints programmed cell death protein 1 receptor (PD-1), programmed cell death protein 1 ligand (PD-L1) and cytotoxic T lymphocyte-associated protein 4 (CTLA-4).

Even though immunotherapy is at the forefront of cancer treatment, many tumors remain immune resistant. Immune based therapies (and specifically tumor vaccines) are frequently constrained by intrinsic tumor cell mechanisms enabling immune privilege/evasion.

Therefore, there is a need in the field to identify additional mechanisms that suppress the immune response during the course of cancer treatment.

### SUMMARY OF THE INVENTION

The present disclosure provides a method for treating a cancer in a subject, wherein the cancer expresses and/or secrets apolipoprotein E (apoE), the method comprising inhibiting expression or activity of apoE, thereby treating the cancer in the subject.

In some embodiments, the method comprises administering to the subject a therapeutically effective amount of an agent that inhibits expression or activity of apoE.

In some embodiments, the agent that inhibits expression of apoE is selected from the group consisting of an RNAi, an siRNA, an miRNA, an antisense oligo- or polynucleotide, a guide RNA, and a polynucleotide coding sequence thereof on a vector.

In some embodiments, the method comprises administering to the subject a therapeutically effective amount of an agent that inhibits activity of apoE.

In some embodiments, the agent that inhibits activity of apoE comprises a peptide, protein or a small molecule that binds to apoE or a receptor of apoE (such as lrp1, lrp8 and/or ldlr).

In some embodiments, the protein that inhibits activity of apoE is an anti-apoE or apoE receptor antagonist/neutralizing antibody or an antigen-binding fragment thereof.

In some embodiments, the anti-apoE or anti-apoE receptor antibody or an antigen-binding fragment thereof is a monoclonal antibody, a humanized antibody, an Fab, Fab′, F(ab′)2, Fd, single chain Fv or scFv, disulfide linked Fv, V-NAR domain, IgNar, intrabody, IgGΔCH2, minibody, F(ab′)3, tetrabody, triabody, diabody, single-domain antibody, DVD-Ig, Fcab, mAb2, (scFv)2, or scFv-Fc.

In some embodiments, the cancer is a solid tumor.

In some embodiments, the cancer is selected from esophageal carcinoma, lung squamous cell carcinoma, lung adenocarcinoma, cervical squamous cell carcinoma, endometrial adenocarcinoma, bladder cancer, urothelial carcinoma, lung cancer, non-small cell lung cancer, colorectal cancer, rectal cancer, endometrial cancer, skin cancer, head & neck squamous cell carcinoma, brain cancer, glioblastoma multiforme, non-CNS tumor, breast cancer, gastric cancer, gastroesophageal cancer, gastroesophageal adenocarcinoma, pancreatic cancer, prostate cancer, ovarian cancer, melanoma, nasopharyngeal carcinoma, anal carcinoma, mesothelioma, renal cell carcinoma, gallbladder/cholangiocarcinoma, pancreatic carcinoma, penile squamous cell carcinoma, and vulvovaginal carcinoma.

In some embodiments, the cancer is melanoma.

In some embodiments, the subject is human.

In some embodiments, the subject is administered a second anti-cancer therapy.

In some embodiments, the second anti-cancer therapy is selected from the group consisting of surgery, radiation, adoptive cell therapy (e.g., CAR T cell therapy), a cancer vaccine, a chemotherapeutic agent, an anti-angiogenesis agent, a growth inhibitory agent, an immunotherapy, and/or an anti-neoplastic composition.

In some embodiments, the second anti-cancer therapy comprises administering an immune checkpoint inhibitor.

In some embodiments, the immune checkpoint inhibitor is an anti-CTLA4, anti-PD1 or anti-PDL1 inhibitor (e.g., antibody or antigen-binding fragment thereof).

In some embodiments, the agent is administered intravenously, subcutaneously, or intratumorally.

It should be understood that any one of the embodiments described herein, including embodiments described only in the examples or claims, can be combined with any one or more other embodiments, unless such combination is not proper or explicitly disclaimed.

### BRIEF DESCRIPTION OF THE FIGURES

FIGS. 1A-1G show that apoE is the most highly expressed immune suppressive gene in the melanoma B16-F10 cell lines and apoE serum levels rise with tumor growth in vivo. FIG. 1A: mRNA expression as revealed by nanostring nsolver Pancancer immune profiling, of the immune suppressive marker genes in B16F10 cells (n=6). FIG. 1B: ApoE expression was undetectable in the serum of apoE knockout (apoE^{-/-}) mice, but present at high levels in wild type (C57/BL6) mice with ELISA assay. FIG. 1C: ApoE knockout mice were inoculated with 10\hat{ }4 wild type (B16F10) cells and serum levels of apoE increased over time with increasing tumor size. FIG. 1D: Validation of apoE gene knockout in B16F10 cells by CRISPR-Cas9 gene deletion. The level of apoE protein expression was measured in WT B16F10 and the corresponding apoE^{-/-} clone by Western Blot. FIG. 1E: Equivalent numbers of WT and apoE^{-/-} B16 cells were plated and apoE levels released into culture media were quantified by ELISA at 48 hr. ApoE is secreted at high levels in WT B16 cells and is not detectable in the apoE^{-/-} cell line. FIG. 1F: To evaluate whether targeting apoE influenced cell viability and proliferation, 5×10\hat{ }4 WT (n=6) or apoE^{-/-} B16 cells (n=6) were grown in 12-well plates and proliferation rates were measured at 24 hr and 48 hr by MTT assay. There is no statistically significant difference in cell proliferation rate between the two groups. FIG. 1G: Cell cycle distribution was determined in WT and apoE^{-/-} B16 cells. The various phases of the cell cycle are differentiated in the flow cytometry plot on the left: G0-G1 is the pre-synthesis phase, S-phase cells are undergoing active DNA synthesis and G2-M cells are preparing for mitosis. Bar graphs represents the percentage of cells in G0-G1, S and M phase of the cell cycle. Cell counts and cell cycle distribution indicate that WT and apoE^{-/-} B16 cells proliferate at equivalent rates. Data are representative of three independent experiments. Results are expressed as mean score±SD. *p<0.05; **p<0.005; ***p<0.001, determined by unpaired two-tailed Student's t-test.

FIGS. 2A-2E show apoE secreted into media from B16 melanoma tumor cells inhibits T-cell proliferation and function. FIG. 2A: Conditioned media (CM) from WT or apoE^{-/-} cells was cultured with T-cells activated by CD3/CD28 beads. Bar graphs depict IFNγ released under these conditions. The production of IFNγ was significantly suppressed when T cells were cultured in WT B16 CM, but this suppression was reversed in apoE free CM from apoE^{-/-} cells. FIG. 2B: Representative flow cytometry plots; FIG. 2C: quantification of T cell apoptosis in CM. Results show that T cell viability is markedly reduced in the WT CM, whereas induction of apoptosis was reversed when cells were cultured in the apoE^{-/-} medium, similar to RPMI control medium alone. FIG. 2D: Representative flow cytometry plots; FIG. 2E: quantification of cell cycle analysis of T cells in different media. Cell cycle distribution showed an arrest of activated T cells in the G0-G1 phase in the WT B16 medium, while in the apoE^{-/-} medium most T cells were distributed in the S and G2-M phases, similar to the RPMI media control. Results are expressed as mean score±SD. *p<0.05; **p<0.005; ***p<0.001, determined by unpaired two-tailed Student's t-test.

FIGS. 3A-3C show that apoE agonist peptide COG133 inhibits cytokine secretion induced by immunogenic tumor cells, while anti-APOE blocking antibody enhances cytokine secretion in tumor cell/splenocyte reactions. FIG. 3A: Naïve and vaccinated splenocyte production of IFNγ was markedly reduced when splenocytes were co-cultured with immunogenic (BET/JQ1 treated) B16 tumor cells in the presence of the apoE agonist COG133. FIG. 3B: Naïve splenocyte production of IFNγ was significantly increased when co-cultured with immunogenic B16 tumor cells with anti-APOE antibody. FIG. 3C: COG133 was also inhibitory of IFNγ production when vaccinated splenocytes were used for co-culture with treated immunogenic cancer cells. It is of interest to note that the levels of IFNγ production is significantly greater when vaccinated splenocytes were used for this experiment. NS: naïve splenocytes, splenocytes were collected from naïve C57BL/6 mice. Treated B16: B16 cells were expose to Myc inhibitor (0.25 μM BET and 0.25 μM JQ1) for 4 days, to induce immunogenicity. VS: vaccinated splenocytes. Irradiated 10⁴ WT B16 tumor cells and 100ug/ml anti-CTLA4 antibody were administered to C57BL/6 mice on day 0, and splenocytes were collected at day 7 after tumor cell inoculation. Data are representative of three independent experiments. Results are expressed as mean score±SD. *p<0.05; **p<0.005; ***p<0.001, determined by unpaired two-tailed Student's t-test.

FIGS. 4A-4D show that lrp8 is the most dominant receptor expressed on activated T cells and blocking lrp8 enhanced T-cell activation. Expression of apoE receptors shows predominance of ldlr and lrp8 receptors on T cells (FIG. 4A), lrp8, ldl and lrp1 receptors on dendritic cells (FIG. 4B), and lrp1 receptors on macrophages as shown by quantitative real-time PCR (qPCR) (FIG. 4C). Expression of ldlr and lrp8 was upregulated following T cell stimulation with CD3/CD28 beads. Expression of lrp8 was also upregulated following dendritic cell stimulation with TLR7/8. FIG. 4D: The amount of IFNγ production from vaccinated splenocytes co-cultured with immunogenic WT B16 cells was quantified by ELISA assay. Results show that IFNγ production was significantly suppressed in the presence of the apoE agonist COG133, but suppression did not occur when splenocytes were harvested from lrp8^{-/-} mice. These findings suggest that T-cell function is at least partially inhibited by apoE through the lrp8 receptor pathway. Results are expressed as mean score±SD. *p<0.05; **p<0.005; ***p<0.001, determined by unpaired two-tailed Student's t-test.

FIGS. 5A-5B show that knock out of apoE in both B16 tumor cells and in mice enhances host immunity and attenuates tumorigenicity. FIG. 5A: In vivo, 10⁴ WT or apoE^{-/-} B16 cells were injected into the right leg of WT (n=9), apoE^{-/-} (n=9) or lrp8^{-/-} mice (n=9). The average tumor growth in each group (n=6) is compared. Tumor growth was significantly slower when apoE^{-/-} B16 cells were injected into apoE^{-/-} or lrp8^{-/-} mice versus the other groups (two-way ANOVA; P<0.0001). FIG. 5B: The final tumor volume between treatment groups at the end point of the experiment (Day 20) was also compared using a one-way ANOVA followed by Tukey HSD pairwise multiple comparisons between treatment groups. Tumor volumes at the end point of the experiment were significantly different between treatment groups (one-way ANOVA; P<0.0001).

FIGS. 6A-6B show survival curve of WT, apoE^{-/-} or lrp8^{-/-} mice injected with WT or apoE^{-/-} B16 cells. FIG. 6A: Survival was significantly better (n=9) when apoE^{-/-} B16 cells were injected into apoE^{-/-} or lrp8^{-/-} mice versus the other groups. FIG. 6B: The median survival time and the cumulative survival probability were calculated and compared using the Kaplan-Meier survival estimator followed by a log-rank test, and the hazard ratio (HR) was calculated using the Cox proportional-hazards regression model. The comparison between the groups is shown in the table.

FIGS. 7A-7B show that the first three tumors that reached 15 mm in each of the experimental groups were harvested and the immune cell infiltrate and immune pathway activation was analyzed with nanostring Pancancer immune profiling. Immune cell type scores (FIG. 7A) and immune pathway (signature) scores (FIG. 7B) show that immune cell infiltrates and immune pathway activation were greatest when apoE^{-/-} cells were injected into apoE^{-/-} mice. Dot line plots show the score trends of 12 immune cell lines and 29 immune pathways. Box plots show selective representative score comparisons. Results are expressed as mean score±SD. *p<0.05; **p<0.005; ***p<0.001, determined by unpaired two-tailed Student's t-test. Wt/wt: wt B16 cells injected in wt mice; apoE^{-/-}/apoE^{-/-}: apoE^{-/-} cells injected in apoE^{-/-} mice; apoE^{-/-}/lrp8^{-/-}: apoE^{-/-} cells injected in lrp8^{-/-} mice; apoE^{-/-}/wt: apoE^{-/-} cells injected in wt mice; wt/apoE^{-/-}: wt cells injected in apoE^{-/-} mice; wt/lrp8^{-/-}: wt cells injected in lrp8^{-/-} mice.

FIGS. 8A-8B show Nanostring PanCancer Immune Profiling analysis of RNAseq of activation markers for T cells and DCs that infiltrated into the tumors from the 6 different groups. Activated T cell genes (FIG. 8A) and activated DC genes (FIG. 8B) were all statistically significantly increased in the tumor from apoE^{-/-}/apoE^{-/-} group when compared with wt/wt control groups. The relative gene expression level was indicated on the y-axis and tumor groups are listed along the x-axis. Results are expressed as mean score±SE. *p<0.05; **p<0.005; ***p<0.001, determined by unpaired two-tailed Student's t-test.

FIGS. 9A-9D show that, to validate nanostring results, both CD45 and CD3 expression were examined with IHC staining in the tumors from WT or apoE^{-/-} mice following injection with WT or apoE^{-/-} tumor cells. Representative images of CD45 (FIG. 9A) and CD3 (FIG. 9C) staining visualized with DAB (brown) and counterstained with hematoxylin (nuclei). FIGS. 9B and 9D show optical density (mean gray value) obtained by color deconvolution analysis. Optical density graph bars represent the mean±SD (n=30 images). *p<0.05; **p<0.005; ***p<0.001, determined by unpaired two-tailed Student's t-test. Wt/wt: wt B16 cells injected in wt mice; apoE^{-/-}/apoE^{-/-}: apoE^{-/-} cells injected in apoE^{-/-} mice,

FIGS. 10A-10B show that apoE knock out in B16 tumor cells induces potent immunogenicity. Wild type and apoE^{-/-} mice (FIG. 10A) or wild type and lrp8^{-/-} mice (FIG. 10B), were inoculated with either WT B16 or apoE^{-/-} cells, with anti-CTLA4 antibody on day 0. Splenocytes were harvested on day 7 and co-cultured with either WT B16 or apoE^{-/-} B16 cells for 48 hr, following which IFN□ levels in media were compared with ELISA assay. Results show that IFN□ production is highest for all groups when apoE^{-/-} cells are used. These findings re-iterate the potent inhibitory effect of apoE on immune cell activation in the cancer environment. Results are expressed as mean score±SD. *p<0.05; **p<0.005; ***p<0.001, determined by unpaired two-tailed Student's t-test.

FIG. 11 shows that ApoE RNA-seq expression is abundant in cutaneous melanoma but is not associated with PD-L1 or PD1. Scatterplots showing mRNA expression correlation of APOE (x-axis) with PD-L1, PD1 and APOC1 from the TCGA melanoma datasets based on their RNA-seq gene expression values (measured by RSEM algorithm). APOE expression did not correlate with PD-L1 or PD1 but did positively correlate with APOC1 expression which was used as a control gene. The correlation was evaluated by the Spearman correlation coefficient with a cut-off value of 0.5 and P-value used a cut-off value of 0.05.

FIG. 12 shows scatterplots showing the tumor purity-corrected partial Spearman's rho value and the correlation between gene expression with infiltration of six immune cell estimates. Top row is APOE expression while the bottom row depicts PD-L1. PD-L1 correlated with CD8 T cell and neutrophil infiltrates, while APOE does not correlate with immune cell infiltrates. The correlation was evaluated by the Spearman correlation coefficient with a cut-off value of 0.5 and P-value used a cut-off value of 0.05.

FIG. 13 shows cumulative survival of patients associated with bifurcate gene expression at 30%. TCGA database includes primary and metastasis samples (n=462). APOE expression did not associate with survival, while PD-L1 and PD1 was associated with survival at this level of analysis. The correlation was evaluated by the Spearman correlation coefficient with a cut-off value of 0.5 and P-value used a cut-off value of 0.05.

FIG. 14 shows that apoE has a repressive effect on T cell function and viability. FIG. 14A: T cells isolated from naïve mouse spleens were cultured in RPMI media, WT B16 conditioned media and apoE^{-/-} B16 conditioned media for 48 hr and the media was analyzed with ProcartaPlex multiplex immunoassay. Results show the production of pro-inflammatory cytokines and chemokines such as LIF, MIP-1α, TNFα, IL18, GM-CSF and IL-13 were suppressed while production of IL-6, RANTES and Gro-α KC were enhanced when T cells were cultured in WT B16 conditioned media. Remarkably, these effects were reversed by incubating T-cells with conditioned media from apoE^{-/-} cells, similar to T-cells activated in control RPMI media alone. Results are expressed as mean score±SD. *p<0.05; **p<0.005; ***p<0.001, determined by unpaired two-tailed Student's t-test. FIG. 14B: The effect of apoE agonist peptide COG133 on the viability of activated mouse T cells was tested by culturing the cells in the presence of the indicated concentrations of peptide for 48 hr and cell death was quantified with flow cytometry using the APC-conjugated Sytox Red dead cell stain. T-cell viability decreases in a dose dependent fashion in the presence of COG133 ApoE agoist. The number in Quadrant 2 is the percentage of dead cells.

FIG. 15 shows role of apoE on effector function of dendritic cells. Mouse primary bone-marrow derived dendritic cells (DC) were cultured in the presence of conditioned medium (CM) from WT B16 and apoE^{-/-} cells for 48 hr with or without toll like receptor (TLR7/8) stimulation. Multiplex ELISA assay was used to detect cytokines and chemokines and results showed that WT B16 CM enhances the production of anti-inflammatory cytokine IL-10 while downregulating the production of proinflammatory cytokines IL1α, IL1β, MIP-1α and MIP-1β, IL28 and RANTES. This effect was reversed when DCs were cultured in apoE−/− cell CM.

FIG. 16 shows that dendritic cell function is modulated by apoE agonist COG133. Mouse bone-marrow derived dendritic cells (DC) were cultured in the presence of the indicated concentrations of apoE agonist COG133 for 48 hr. COG133 increased the production of anti-inflammatory IL-10, GM-CSF, and chemokines MCP-1 and MCP-3 by TLR7/8 activated DC while decreasing the levels of proinflammatory cytokines IL-1α, IL-1β and IL-23 in a dose-dependent manner as determined by multiplex ELISA.

FIG. 17 shows that vaccination with immunogenic WT B16 tumor cells enhances splenocyte response which is dampened by the presence of apoE agonist COG 133. Splenocytes from naïve mice (NS) as well as mice vaccinated with 104 WT B16 and 100 μg/ml anti-CTLA4 antibody (VS) were co-cultured for 48 hr with either WT B16 cells or Myc-inhibited immunogenic B16 tumor cells in the presence of the indicated concentrations of apoE agonist COG133. Multiplex ELISA shows the suppressive effect of the apoE agonist in production of IFNγ, IL-6 and IL-18 in the cocultures with naïve splenocytes and this suppressive effect although diminished in vaccinated splenocytes is still observed.

FIG. 18 shows that ApoE affects T cell function at least partially through lrp8 receptor pathway. Splenocytes were isolated from vaccinated WT mice as well as lrp8−/− mice and cocultured for 48 hr with Myc-inhibited immunogenic B16 cells, in the presence of the indicated concentrations of apoE agonist COG133. Multiplex immunoassay shows a suppression of proinflammatory cytokines and chemokines IL-13, IL-4, IL22, IL18, MCP-1, RANTES and Gro-α in the cocultures with WT vaccinated splenocytes. However, this suppressive effect is diminished in lrp8^{-/-} vaccinated splenocytes.

FIGS. 19A-19D show that neuroblastoma (Neuro2a) and melanoma (B16F10) tumor exhibit distinct immune characteristics. Tumors (n=3) were harvested when they reach 15 mm and immune-phenotyped with Nanostring nsolver Pancancer immune platform. FIG. 19A: Trend plot of infiltrating immune cell type scores. FIG. 19B: Box plots of typical genes associated with T cells, macrophages, neutrophils, NK cells and total TILs are shown. Melanoma tumors exhibit statistically significantly lower numbers of exhausted CD8 T cells, whereas infiltration of other cell types were similar. FIG. 19C Trend plot of immune pathway scores. FIG. 19D: Pathway scores were presented as box plots for select immune pathways of interest. All pathways were significantly decreased in melanoma tumors when compared to the neuroblastoma tumors suggesting suppression of immune activation in melanoma. Unpaired two-tailed Student's t-test was performed for statistical analysis (p<0.05).

FIGS. 20A-20B show that apolipoprotein E (apoE) expression is highly expressed in melanoma tumor cells. Immune gene profiles were compared between neuroblastoma (Neuro2A) and melanoma (B16F10) cell lines with Nanostring nsolver Pancancer immune platform. FIG. 20A: List of top 11 genes with an immune-suppressive phenotype between the cell lines. ApoE is 7,540-fold greater in the melanoma (B16F10) than in the neuroblastoma (Neuro2a) cell line. FIG. 20B Confirmation with real-time quantitative PCR (RT-qPCR) (p<0.005, Student t test).

### DETAILED DESCRIPTION

Before the present disclosure is described, it is to be understood that this disclosure is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present disclosure, the preferred methods and materials are now described. All patents, applications and non-patent publications mentioned in this specification are incorporated herein by reference in their entireties.

### (I) Definitions

As used herein, the term “antibody,” in the broadest sense, encompasses various antibody structures, including but not limited to monoclonal antibodies, polyclonal antibodies, and multispecific antibodies (e.g., bispecific antibodies). The term “antibody” may also broadly refers to a molecule comprising complementarity determining region CDR1, CDR2, and CDR3 of a heavy chain and CDR1, CDR2, and CDR3 of a light chain, wherein the molecule is capable of binding to an antigen. The term “antibody” also includes, but is not limited to, chimeric antibodies, humanized antibodies, human antibodies, and antibodies of various species such as mouse, human, cynomolgus monkey, etc.

As used herein, the term “antibody fragment” or “antigen-binding fragment” includes, but is not limited to, fragments that are capable of binding antigen, such as Fv, single-chain Fv (scFv), Fab, Fab′, and (Fab′)₂. In certain embodiments, an antibody fragment includes Fab, Fab′, F(ab′)₂, Fd, single chain Fv or scFv, disulfide linked Fv, V-NAR domain, IgNar, intrabody, IgGΔCH2, minibody, F(ab′)₃, tetrabody, triabody, diabody, single-domain antibody, DVD-Ig, Fcab, mAb₂, (scFv)₂, or scFv-Fc.

As used herein, the terms “apoE” or “APOE”, also known as “apolipoprotein E,” “Alzheimer's Disease 2,” “LPG” and “LDLCQ5,” refer to the well-known gene that encodes the protein, APOE. APOE is synthesized throughout the body, primarily in the liver and functions as a lipid transport protein. It is found on very-low-density (VLDL), low-density (LDL) and high-density (HDL) lipoprotein particles and mediates their cellular uptake via the B- and E-receptor as well as the LDL receptor-related protein (LRP) receptor. Nucleotide and amino acid sequences of APOE can be found, for example, at GenBank Accession No. NM_000041.4 (Homo sapiens APOE); GenBank Accession No. NM_001270681.1 (Rattus norvegicus APOE); GenBank Accession No. NM_001305843.1 (Mus musculus APOE); GenBank Accession No. XM_028839202.1 (Macaca mulatta APOE); and GenBank Accession No. XM_005589554.2 (Macaca fascicularis APOE).

The human APOE gene contains two single-nucleotide polymorphisms that result in the three most common variants, APOE2, APOE3, and APOE4. The term APOE as used herein also refers to variations of the APOE gene including variants of human APOE.

ApoE is a major ligand for the low density lipoprotein (LDL) receptor, also referred to as ldlr, and also known to bind to other members of the LDL receptor-related protein (LRP) family (lrp1, lrp1b, megalin/lrp2, ldlr, very low-density lipoprotein receptor, megf7/lrp4, lrp8/apolipoprotein E receptor2).

As used herein, the terms “administering,” “administration,” and like terms refer to any method which, in sound medical practice, delivers a composition containing an active agent to a subject in such a manner as to provide a therapeutic effect. Suitable routes of administration can include oral, parenteral, subcutaneous, intravenous, intramuscular, intratumor or intraperitoneal.

As used herein, an “effective” or “therapeutically effective” amount of an agent that inhibits expression or activity of apoE for treating or preventing a cancer, such as an apoE-expressing cancer, is the amount of the agent sufficient to alleviate one or more signs and/or symptoms of the cancer in the treated subject, whether by inducing the regression or elimination of such signs and/or symptoms or by inhibiting the progression of such signs and/or symptoms.

As used herein, the expression “in combination with” means that a first therapeutic agent, e.g., an anti-apoE blocking antibody or antigen-binding fragment thereof, is administered before, after, or concurrent with a second therapeutic agent, e.g., an anti-PD-1, anti-CDLA4 or anti-PD-L1 antibody or antigen-binding fragment thereof. The term “in combination with” also includes sequential or concomitant administration of a first therapeutic agent, e.g., an anti-apoE blocking antibody or antigen-binding fragment thereof, and a second therapeutic agent, e.g., an anti-PD-1, anti-CDLA4 or anti-PD-L1 antibody or antigen-binding fragment thereof.

As used herein, the term “subject” refers to a mammal (e.g., rat, mouse, cat, dog, cow, sheep, horse, goat, rabbit), preferably a human, for example, in need of prevention and/or treatment of a cancer, e.g., an apoE-expressing cancer. The subject may have a cancer, e.g., an apoE-expressing cancer, be predisposed to developing such a condition, and/or would benefit from an inhibition or reduction in apoE expression and/or activity, or a depletion of apoE-expressing cells. In one embodiment, the subject may have, or be at risk of developing, a cancer. In many embodiments, the term “subject” may be used interchangeably with the term “patient.”

As used herein, term “a subject in need thereof” means a human or non-human mammal that exhibits one or more symptoms or indications of cancer, and/or who has been diagnosed with cancer, including a solid tumor and who needs treatment for the same.

As used herein, the term “solid tumor” refers to an abnormal mass of tissue that usually does not contain cysts or liquid areas. Solid tumors may be benign (not cancer) or malignant (cancer). For the purposes of the present disclosure, the term “solid tumor” means malignant solid tumors. The term includes different types of solid tumors named for the cell types that form them, viz. sarcomas, carcinomas and lymphomas. However, the term does not include leukemias. In various embodiments, the term “solid tumor” includes cancers arising from connective or supporting tissue (e.g., bone or muscle) (referred to as sarcomas), cancers arising from the body's glandular cells and epithelial cells which line body tissues (referred to as carcinomas), and cancers of the lymphoid organs such as lymph nodes, spleen and thymus (referred to as lymphomas). Lymphoid cells occur in almost all tissues of the body and therefore, lymphomas may develop in a wide variety of organs. In certain embodiments, the term “solid tumor” includes cancers including, but not limited to, colorectal cancer, ovarian cancer, prostate cancer, breast cancer, brain cancer, cervical cancer, bladder cancer, anal cancer, uterine cancer, colon cancer, liver cancer, pancreatic cancer, lung cancer, endometrial cancer, bone cancer, testicular cancer, skin cancer, kidney cancer, stomach cancer, esophageal cancer, head and neck cancer, salivary gland cancer, and myeloma. In certain embodiments, the term “solid tumor” includes cancers including, but not limited to, hepatocellular carcinoma, non-small cell lung cancer, head and neck squamous cell cancer, basal cell carcinoma, breast carcinoma, cutaneous squamous cell carcinoma, chondrosarcoma, angiosarcoma, cholangiocarcinoma, soft tissue sarcoma, colorectal cancer, melanoma, Merkel cell carcinoma, and glioblastoma multiforme. In certain embodiments, the term “solid tumor” comprises more than one solid tumor lesions located separate from one another, e.g., 2, more than 2, more than 5, more than 10, more than 15, more than 20, or more than 25 lesions in a subject in need of treatment. In certain embodiments, the more than one lesions are located distally from one another in the same organ. In certain other embodiments, the tumor lesions may be located in different organs.

As used herein, the terms “treat”, “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition or method herein needs only to reduce the severity of a disease, disorder, or condition, reduce the severity of symptoms associated therewith, or provide improvement to a patient or subject's quality of life.

### (H) Methods of Treating Cancer

The present disclosure provides methods of treating, preventing and/or ameliorating cancer, as well as methods of improving/enhancing immunotherapy, comprising inhibiting expression or activity of apolipoprotein E (apoE), wherein the cancer expresses and/or secrets apoE.

In some embodiments, the method is a method of treating cancer. In some embodiments, the method is a method of preventing cancer. In some embodiments, the method is a method of ameliorating cancer. In some embodiments, the method is a combination therapy. In some embodiments, the method is an enhanced immunotherapy. In some embodiments, the method is for increasing immune activation. In some embodiments, treating, preventing, ameliorating or enhancing comprises preventing immunosuppression to an immune cell, thereby increasing immune activation in the immune cell. In some embodiments, immune activation is activation of an immune cell. Increased activation can be determined by any method known in the art, or with any known activation marker.

A cancer, for the purposes herein, refers to a disease characterized by abnormal, excessive and/or uncontrolled cell growth. Exemplary cancers that can be treated by the methods of the disclosure include, but are not limited to, esophageal carcinoma or esophageal cancer, lung squamous cell carcinoma, lung adenocarcinoma, cervical squamous cell carcinoma or cervical carcinoma, glioma, thyroid cancer, lung cancer (e.g., non-small cell lung cancer), colorectal cancer, colon cancer, bladder cancer, rectal cancer, head and neck cancer, stomach cancer, liver cancer, pancreatic cancer, renal cancer, urothelial cancer, prostate cancer or prostate adenocarcinoma, testis cancer, breast cancer (e.g., ductal or lobular carcinoma), cervical cancer or cervical carcinoma, endometrial cancer, ovarian cancer, gastroesophageal cancer, (e.g., gastroesophageal adenocarcinoma), non-central nervous system (CNS) tumor, melanoma, nasopharyngeal carcinoma, anal carcinoma, mesothelioma, renal cell carcinoma (e.g., chromophobe, clear cell, or papillary), gallbladder/cholangiocarcinoma, pancreatic carcinoma, penile squamous cell carcinoma, or vulvovaginal carcinoma. In one embodiment, the cancer is an EGFR expressing cancer. A wide range of cancers express apoE. Accordingly, the methods of the present disclosure can be used in treating a wide range of cancers.

In some embodiments, the cancer is a solid tumor. In some embodiments, the cancer is esophageal carcinoma, lung squamous cell carcinoma, lung adenocarcinoma, cervical squamous cell carcinoma, endometrial adenocarcinoma, bladder cancer, urothelial carcinoma, lung cancer, non-small cell lung cancer, colorectal cancer, rectal cancer, endometrial cancer, skin cancer, head & neck squamous cell carcinoma, brain cancer, glioblastoma multiforme, non-CNS tumor, breast cancer, gastric cancer, gastroesophageal cancer, gastroesophageal adenocarcinoma, pancreatic cancer, prostate cancer, ovarian cancer, melanoma, nasopharyngeal carcinoma, anal carcinoma, mesothelioma, renal cell carcinoma, gallbladder/cholangiocarcinoma, pancreatic carcinoma, penile squamous cell carcinoma, or vulvovaginal carcinoma.

In some embodiments, the cancer is a skin cancer. In some embodiments, the cancer is melanoma.

In some embodiments, the cancer expresses and/or secretes apoE. In some embodiments, the cancer cells, e.g., melanoma cells, overexpress apoE and/or secrete more apoE as compared to non-cancerous cells, e.g., normal skin cells.

In some embodiments, the methods comprise identifying level of expression and/or secretion of apoE by the cancer. In some embodiment, the identifying comprising measuring apoE level (e.g., mRNA or protein) from a tumor biopsy or serum of a subject. Methods of assessing expression of a protein is well known in the art (e.g., qPCR, ELISA, immunohistochemistry) and are applicable for the methods of the present disclosure.

In some embodiments, the cancer expresses an immune checkpoint inhibitory molecule. In some embodiments, the cancer overexpresses an immune checkpoint inhibitory molecule. In some embodiments, the immune checkpoint inhibitory molecule is a checkpoint inhibitory ligand. In some embodiments, the immune checkpoint inhibitory molecule is selected from PD-L1, PD-L2, CD80, CD86, CD275, CD276, VTCN1, VISTA, HHLA2, HVEM, FGL1, CD155, CD112, CD200, HMGB1, Butyrophilin family members and Gal-9. In some embodiments, the cancer is a PD-L1 positive cancer. In some embodiments, the cancer is a PD-L2 positive cancer. In some embodiments, the cancer is an HVEM positive cancer.

In some embodiments, the subject is a mammal. In some embodiments, the subject is a human. In some embodiments, the subject suffers from cancer. In some embodiments, the subject is naive to cancer treatment. In some embodiments, the subject is naive to immunotherapy. In some embodiments, the subject has received treatment. In some embodiments, the subject is refractive to treatment. In some embodiments, the treatment is an immunotherapy. In some embodiments, the treatment is the immunotherapy. In some embodiments, the treatment is an immunotherapy other than the immunotherapy. In some embodiments, the treatment is selected from radiotherapy, chemotherapy, targeted therapy and immunotherapy. In some embodiments, the subject was previously administered a therapy and the therapy was found ineffective. In some embodiments, the subject was previously administered a therapy, the therapy was initially effective but has become ineffective or less effective.

### (III) Agents for Inhibiting apoE

In some aspects, the methods of the disclosure comprise administering to the subject in need a therapeutically effective amount of an agent that inhibits expression or activity of apoE.

In some embodiments, inhibiting expression of apoE comprises decreasing mRNA or protein level of apoE. In some embodiments, inhibiting expression of apoE comprises decreasing apoE translation.

In some embodiments, inhibiting expression of apoE comprises administering a nucleic acid molecule that inhibits expression of apoE. In some embodiments, the nucleic acid molecule is a regulatory nucleic acid molecule. In some embodiments, the nucleic acid molecule is a guide RNA (for CRIPSR/Cas system effector enzyme). In some embodiments, inhibiting expression of apoE comprises administering a genome editing protein or complex.

In some embodiments, the regulatory nucleic acid molecule is an RNA. In some embodiments, decreasing expression comprises administering a regulatory RNA (e.g., antisense oligonucleotide or siRNA) that inhibits apoE expression.

In some embodiments, the regulatory nucleic acid molecule is an antisense oligonucleotide or antisense polynucleotide. In some embodiments, the regulatory nucleic acid molecule is a non-naturally occurring nucleic acid molecule. In some embodiments, the regulatory nucleic acid molecule is an artificial nucleic acid molecule. In some embodiments, the regulatory nucleic acid molecule comprises a chemical modification. In some embodiments, the chemical modification is a chemically modified backbone. In some embodiments, the chemically modified backbone comprises at least one of: a phosphate-ribose backbone, a phosphate-deoxyribose backbone, a phosphorothioate-deoxyribose backbone, a 2′-O-methyl-phosphorothioate backbone, a phosphorodiamidate morpholino backbone, a peptide nucleic acid backbone, a 2-methoxyethyl phosphorothioate backbone, a constrained ethyl backbone, an alternating locked nucleic acid backbone, a phosphorothioate backbone, N3′-P5′ phosphoroamidates, 2′-deoxy-2′-fluoro-P-d-arabino nucleic acid, cyclohexene nucleic acid backbone nucleic acid, tricyclo-DNA (tcDNA) nucleic acid backbone, ligand-conjugated antisense and a combination thereof. Each possibility represents a separate embodiment of the invention.

In some embodiments, the regulatory nucleic acid molecule binds to apoE mRNA. In some embodiments, the molecule binds to the apoE genomic locus. In some embodiments, the molecule targets a genome editing protein or complex to the APOE genomic locus. In some embodiments, the regulatory molecule binds to a APOE regulatory domain. In some embodiments, the regulatory domain is the promoter. In some embodiments, the regulatory molecule is in a pharmaceutical composition.

In some embodiments, the regulatory RNA is a short inhibitory RNA (siRNA). In some embodiments, the regulatory RNA is RNAi. In some embodiments, the regulatory RNA is a short hairpin RNA (shRNA). In some embodiments, the regulatory RNA is microRNA (miR).

In some embodiments, the regulatory molecule is introduced to a cell through an expression vector. In some embodiments, the expression vector is configured for expression in the cancer cell. In some embodiments, the expression vector is configured for expressing the regulatory molecule. In some embodiments, the vector comprises a polynucleotide sequence encoding the regulatory RNA that inhibits apoE expression.

In some embodiments, decreasing expression comprises administering a pharmaceutical composition. In some embodiments, the pharmaceutical composition comprises the regulatory molecule. In some embodiments, the pharmaceutical composition comprises an expression vector comprising a polynucleotide sequence encoding the regulatory molecule.

In some embodiments, genome editing protein edits the APOE genomic locus such that apoE is no longer expressed. In some embodiments, genome editing protein ablates a portion of the apoE genomic locus. In some embodiments, genome editing protein introduces a mutation into the apoE genomic locus. In some embodiments, the genome editing protein introduces a deletion into the apoE genomic locus. Genome editing proteins/complexes are well known in the art and any such protein/complex may be employed for editing the apoE genomic locus. In some embodiments, a genome editing protein is selected from the group consisting of a clustered Regularly Interspaced Short Palindromic Repeats (CRISPR)-associated nuclease, a Zinc-finger nuclease (ZFNs), a meganuclease and a transcription activator-like effector nuclease (TALEN). In some embodiments, the decreasing comprises expressing the genome editing protein in the subject. In some embodiments, the decreasing comprises expressing the genome editing protein in the cancer cell.

Expressing of a molecule within a cell is well known to one skilled in the art. It can be carried out by, among many methods, transfection, viral infection, or direct administration. In some embodiments, the gene is in an expression vector such as plasmid or viral vector. In some embodiments, the vector is with in an appropriate composition for allowing entrance of the vector into cells. For example, placing the vector within a liposome or micelle will allow for uptake of the vector into cells.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence. The vector may be a DNA plasmid delivered via non-viral methods or via viral methods. The viral vector may be a retroviral vector, a herpes viral vector, an adenoviral vector, an adeno-associated viral vector or a poxviral vector. The promoters may be active in mammalian cells. The promoters may be a viral promoter. The promoter may be active in cancer cells.

In some embodiments, the polynucleotide encoding the regulatory RNA is operably linked to a promoter. The term “operably linked” is intended to mean that the nucleotide sequence of interest is linked to the regulatory element or elements in a manner that allows for expression of the nucleotide sequence (e.g. in an in vitro transcription/translation system or in a host cell when the vector is introduced into the host cell).

In some embodiments, the vector is introduced into the cell by standard methods including electroporation (e.g., as described in From et al., Proc. Natl. Acad. Sci. USA 82, 5824 (1985)), Heat shock, infection by viral vectors, high velocity ballistic penetration by small particles with the nucleic acid either within the matrix of small beads or particles, or on the surface (Klein et al., Nature 327. 70-73 (1987)), and/or the like.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMTI, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma vims include pBV-lMTHA, and vectors derived from Epstein Bar vims include pHEBO, and p205. Other exemplary vectors include pMSG, pAV009/A+, pMTO10/A+, pMAMneo-5, baculovims pDSVE, and any other vector allowing expression of proteins under the direction of the SV-40 early promoter, SV-40 later promoter, metallothionein promoter, murine mammary tumor vims promoter, Rous sarcoma vims promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et ah, Molecular Cloning: A Laboratory Manual, Cold Springs Harbor Laboratory, New York (1989, 1992), in Ausubel et ah, Current Protocols in Molecular Biology, John Wiley and Sons, Baltimore, Md. (1989), Chang et ah, Somatic Gene Therapy, CRC Press, Ann Arbor, Mich. (1995), Vega et ah, Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polynucleotide.

In certain other embodiments, inhibiting activity of apoE comprises administering an agent that binds to apoE or a receptor of apoE (e.g., lrp1, lrp8, ldlr).

In some embodiments, the agent binds to soluble apoE. In some embodiments, binding of the agent induces degradation of apoE. In some embodiments, binding of the agent reduces and/or inhibits apoE function. In some embodiments, binding of the agent blocks binding of apoE to a receptor.

In some embodiments, the agent binds to a receptor of apoE. In some embodiments, the receptor is a low-density lipoprotein (LDL) receptor, ldlr. In some embodiments, the receptor is a LDL receptor-related protein (LRP) receptor. In some embodiments, the agent binds to lrp1, lrp8 or ldlr receptor. In some embodiments, binding of the agent to blocks binding of the receptor to apoE. In some embodiments, binding of the agent inhibits function of the receptor.

In some embodiments, the agent is in a pharmaceutical composition. In some embodiments, the agent is a peptide, a protein or a small molecule (e.g., with M.W. less than about 15 Da, 1200 Da, 1000 Da, 800 Da, 500 Da, or 250 Da).

In some embodiments, the agent is a small molecule inhibitor.

In some embodiments, the protein that inhibits activity of apoE is an anti-apoE or apoE receptor antagonist, a neutralizing antibody or an antigen-binding fragment thereof.

In some embodiments, the agent is an antibody. In some embodiment, the monoclonal antibodies of the invention or antigen-binding fragments thereof are human-mouse chimeric antibodies, humanized antibodies, human antibodies, CDR-grafted antibodies, or resurfaced antibodies.

In certain embodiments, the antibody is an IgG, such as IgG1, IgG2, IgG3, or IgG4.

In some embodiments, the antigen-binding fragment thereof is an Fab, Fab′, F(ab′)₂, F_{d}, single chain Fv or scFv, disulfide linked Fᵥ, V-NAR domain, IgNar, intrabody, IgGΔCH2, minibody, F(ab′)₃, tetrabody, triabody, diabody, single-domain antibody, DVD-Ig, Fcab, mAb₂, (scFv)₂, or scFv-Fc.

The agent that inhibits expression or activity of apoE of the present disclosure may be administered by any delivery route which results in a therapeutically effective outcome. In some embodiments, the agent is administered intravenously, subcutaneously, intraperitioneally, or intratumorally.

### (IV) Combination Therapies

The present disclosure provides methods of treating and preventing a disease, e.g., a cancer, by administering a therapeutically effective amount of an agent that inhibits expression or activity of apoE as a monotherapy, and optionally with one or more anti-cancer therapy, e.g., a second therapeutic agent or therapy.

In certain embodiments, the second therapeutic agent or therapy is selected from surgery, radiation, chemotherapy (e.g., anti-cancer chemotherapy, for example, paclitaxel, docetaxel, vincristine, cisplatin, carboplatin or oxaliplatin), CAR-T cell therapy, a cancer vaccine, an oncolytic virus, a cytokine, an anti-angiogenesis agent (e.g., anti-VEGF therapy), a growth inhibitory agent (e.g., anti-EGFR therapy), an anti-cancer drug, an immunotherapy, and/or an anti-neoplastic composition. As used herein, “anti-cancer drug” means any agent useful to treat cancer including, but not limited to, cytotoxins and agents such as antimetabolites, alkylating agents, anthracyclines, antibiotics, antimitotic agents, procarbazine, hydroxyurea, asparaginase, corticosteroids, mytotane (O,P′-(DDD)), biologics (e.g., antibodies and interferons) and radioactive agents. As used herein, “a cytotoxin or cytotoxic agent”, also refers to a chemotherapeutic agent and means any agent that is detrimental to cells. Examples include Taxol® (paclitaxel), temozolamide, cytochalasin B, gramicidin D, ethidium bromide, emetine, cisplatin, mitomycin, etoposide, tenoposide, vincristine, vinbiastine, coichicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In certain embodiments, the methods comprise administering an agent to reduce or ameliorate or treat symptoms of an immune-related adverse event. In one embodiment, the agent is selected from an IL-6 inhibitor (e.g., an anti-IL6 receptor antibody such as tocilizumab or sarilumab), a corticosteroid or a non-steroidal anti-inflammatory agent.

In some embodiments, the second anti-cancer agent or therapy is an immunotherapy. In some embodiments, the immunotherapy is a therapy that does not inhibit apoE expression or activity. In some embodiments, the immunotherapy is a different immunotherapy.

In some embodiments, the immunotherapy is a targeted immunotherapy. As used herein, the term “targeted immunotherapy” refers to an immunotherapy that strives to induce a specific immune response to a tumor antigen. A target immunotherapy therefore induces an immune response targeted to the cancer. Examples of targeted immunotherapy include, but are not limited to, cytotoxic antibodies and chimeric antigen receptor (CAR) expressing cells with a CAR that targets a tumor antigen. Examples of tumor antigens that are currently targeted by immuno therapeutics include, for example, CD52 (alemtuzumab), EGFR (cetuximab/panitumumab), CD20 (rituximab), MUC1, and Her2 (trastuzumab).

In some embodiments, the immunotherapy is an immune cell transplant. In some embodiments, the immunotherapy is adoptive T cell therapy. In some embodiments, the T cell is a tumor infiltrating lymphocyte (TIL). In some embodiments, the T cell is a CD8 positive T cell. In some embodiments, the T cell is a cytotoxic T cell. In some embodiments, the immune cell is an autologous immune cell. In some embodiments, the immune cell is non-autologous immune cell. In some embodiments, the immune cell is a syngeneic immune cell. In some embodiments, the immune cell is an allogenic immune cell. In some embodiments, the immune cell is peripheral blood mononuclear cells (PBMCs). In some embodiments, the immune cell is selected from a T cell, a B cell, a macrophage, and a natural killer cell (NK cell). In some embodiments, the immune cell is selected from a T cell, a macrophage, and a natural killer cell (NK cell). In some embodiments, the immune cell is a T cell. In some embodiments, the immune cell is a CAR cell. In some embodiments, the CAR cell is selected from a CAR-T cell and a CAR-NK cell.

In some embodiments, the immunotherapy is immune checkpoint blockade. Immune checkpoint blockade is well known in the art and is a method for activating immune surveillance that may have been avoided by a cancer expressing an immune inhibitory ligand. The blockade may activate immune cells, increase immune cell proliferation, make the tumor cells detectable by the immune system or a combination of the above. In some embodiments, immune checkpoint blockade is immune checkpoint inhibition. In some embodiments, immune checkpoint blockade comprises administering an immune checkpoint inhibitor, e.g., antibody that binds to an immune checkpoint protein. In some embodiments, the antibody is in a pharmaceutical composition. In some embodiments, the checkpoint protein is a receptor on the immune cell. In some embodiments, the checkpoint protein is a ligand expressed by the cancer cell. In some embodiments, the antibody inhibits the immune checkpoint protein. In some embodiments, the immune checkpoint protein is selected from PD-1, PD-L1, PD-L2, CD80, CD86, VISTA, CD275, CD276, VTCN1, HHLA2, CD96, CD155, TIGIT, CD112R, CD112, CD200, CD200R, CTLA4, LAG3, FGL1, TIM3, CEACAM-1, Gal-9, HMGB1, Butyrophilin family members, HVEM, and BTLA. In some embodiments, the immune checkpoint protein is PD-1. In some embodiments, the immune checkpoint protein is PD-L1. In some embodiments, the immune checkpoint protein is CTLA4.

In some embodiments, the immunotherapy is anti-PD-1 therapy. In some embodiments, the immunotherapy is anti-PD-1 antibody therapy. In some embodiments, the immunotherapy is anti-PD-1 immune checkpoint blockade. In some embodiments, the immunotherapy is PD-1 blockade. In some embodiments, the immunotherapy is anti-PD-1/PD-L1/PD-L2 therapy. In some embodiments, the immunotherapy is anti-PD-1/PD-L1/PD-L2 antibody therapy. In some embodiments, the immunotherapy is anti-PD-1/PD-L1/PD-L2 immune checkpoint blockade. In some embodiments, the immunotherapy is PD-1/PD-L1/PD-L2 blockade. In some embodiments, the immunotherapy is anti-PD-1/PD-L1 therapy. In some embodiments, the immunotherapy is anti-PD-1/PD-L1 antibody therapy. In some embodiments, the immunotherapy is anti-PD-1/PD-L1 immune checkpoint blockade. In some embodiments, the immunotherapy is PD-1/PD-L1 blockade.

In some embodiments, the second anti-cancer therapy comprises administering an immune checkpoint inhibitor. In some embodiments, the immune checkpoint inhibitor is an anti-CTLA4, anti-PD1 or anti-PDL1 inhibitor (e.g., antibody or antigen-binding fragment thereof).

In some embodiments, the immunotherapy comprises one of the therapies provided hereinabove. In some embodiments, the immunotherapy is a combination immunotherapy comprising at least two immunotherapies. In some embodiments, the combination immunotherapy is immune checkpoint blockade and immune cell transfer. In some embodiments, the immunotherapy is administered to the subject. In some embodiments, the immune checkpoint inhibitor is administered to the subject. In some embodiments, the immunotherapy is applied to ex vivo immune cells. In some embodiments, ex vivo immune cells are treated with the immunotherapy. In some embodiments, the ex vivo immune cells are treated with an immune checkpoint inhibitor that binds to an immune receptor on the immune cells. It will be understood by a skilled artisan that when the checkpoint inhibitor is administered to the ex vivo cells to be transferred, it cannot be a molecule that binds the ligand on the cancer cells, but rather must target the receptor on the immune cells. In some embodiments, the treated immune cells are administered to the subject. In some embodiments, the immune cells are in a pharmaceutical composition. In some embodiments, the pharmaceutical composition is administered to the subject. In some embodiments, the administering the treated immune cells is adoptive T cell therapy.

In some embodiments, inhibiting expression or activity of apoE improves the efficacy of the immunotherapy. In some embodiments, improving efficacy is enhancing the immunotherapy. In some embodiments, enhancement or improvement comprises at least a 10, 20, 25, 30, 40, 50, 60, 70, 75, 80, 90, 100, 150, 200, 250, 300, 350, 400, 450, or 500% increase in efficacy. Each possibility represents a separate embodiment of the invention. In some embodiments, efficacy is measured by specific cell killing. In some embodiments, cell killing is cancer cell killing. In some embodiments, the increase is in the number of killed tumor cells. In some embodiments, efficacy is measured in tumor size. In some embodiments, efficacy is measured in time until relapse. In some embodiments, improved efficacy or enhancement is converting a non-effective immunotherapy to an effective immunotherapy. In some embodiments, improved efficacy or enhancement is converting an immunotherapy to which the patient is refractive to a once again effective therapy.

### EXAMPLES

### Example 1: Overview

The need for more effective therapy of tumors like neuroblastoma and melanoma is evident in the poor outcomes of high-risk or advanced disease. Cancer vaccines and immune-based therapies hold great promise for malignant solid tumors, but despite robust immune activation with targeted checkpoint inhibitors, cure is often elusive. Immune based therapies (and specifically tumor vaccines) are frequently constrained by intrinsic tumor cell mechanisms enabling immune privilege/evasion. In the study disclosed herein, tumor secreted apoE was identified as a novel checkpoint enabling immune evasion in a mouse melanoma tumor model.

Recent work has demonstrated that amplification of the Myc oncogene is associated with immune privilege in neuroblastoma. Targeting Myc in vitro with small molecule inhibitors induced tumor cell immunogenicity and enabled whole cell tumor vaccines in mouse tumor models. The vaccine was combined with immune checkpoint inhibitors and induced potent tumor specific immunity. The neuro2a neuroblastoma model is highly sensitive to this whole cell vaccine strategy and despite induction of robust cellular immunity in the B16 melanoma model it remains remarkably resistant. Immune cell infiltrates were similar in both models, but activation of immune signaling pathways were markedly suppressed in the melanoma model. Apolipoprotein E (apoE) had seven thousand five-hundred-fold greater mRNA expression in the melanoma cell line than in the neuroblastoma cell line. ApoE is secreted by the tumors themselves and the level of serum apoE increased dramatically with melanoma tumor growth in apoE KO mice. ApoE is a polymorphic multifunctional protein, classically considered to play a critical role in atherosclerosis and neurodegenerative diseases. Knockout mice fed an atherogenic diet develop pronounced hypercholesterolemia along with an immune-activated phenotype. Experimental models reveal a critical function for myeloid derived apoE modulating DC antigen presentation and T-cell priming. ApoE attenuates inflammation by complex formation with activated C1q, while most recently it was shown that common germline mutations of the human APOE gene modulate melanoma progression and survival.

In tumors, APOE itself is shown to act as an autocrine or paracrine modulator of carcinogenesis. In several human cancers, APOE gene expression is significantly higher in cancer tissue than in adjacent non-cancer tissue and higher levels of serum APOE are associated with metastasis. In pancreatic ductal adenocarcinoma (PDA) patients, elevated plasma APOE protein levels are associated with poor survival, whereas tumor associated macrophages that are key drivers of immunosuppression are characterized by elevated levels of ApoE in both mouse and human PDA. Further studies reveal that apoE KO mice have less orthotopic mammary tumor development and pulmonary metastasis than wild type (WT) mice and lung tumor development and metastasis are suppressed through enhancing anti-tumor activity of natural killer (NK) cells. In contrast, it is reported that apoE is involved in the inhibition of melanoma metastases and has anti-angiogenic properties. ApoE promoted anti-tumor immunity by targeting infiltrating innate myeloid derived suppressor cells (MDSC) via Liver X receptor (LXR) agonism. Furthermore, pretreating cancer cells with apoE inhibited their growth in mouse models. These conflicting observations suggest that the context in which apoE is engaged and the specific APOE genotype in humans may determine its effects, but apoE appears to inhibit its cellular target in most circumstances.

Here we investigate the immunogenic effect of targeting apoE in tumor cells and its immune-modulatory role in the context of tumor growth in the mouse melanoma model. The results reveal a critical role for tumor secreted apoE as a comprehensive checkpoint of both dendritic cell activation and T-cell function. Mechanistically, apoE induces IL-10 secreting suppressive dendritic cells and directly inhibits T-cell function at least partially via the lrp8 receptor. ApoE is a novel checkpoint with extensive and potent suppressant effects on cancer immunity. It is anticipated that targeting apoE will augment immune based therapy in apoE secreting immune resistant tumors.

### Example 2: Tumors in Mouse Models of Neuroblastoma (Neuro2a) and Melanoma (B16-F10) have Similar Immune Cell Infiltrates, but Immune Mediated Pathways are Active in Neuroblastoma and Inactive in Melanoma Tumors

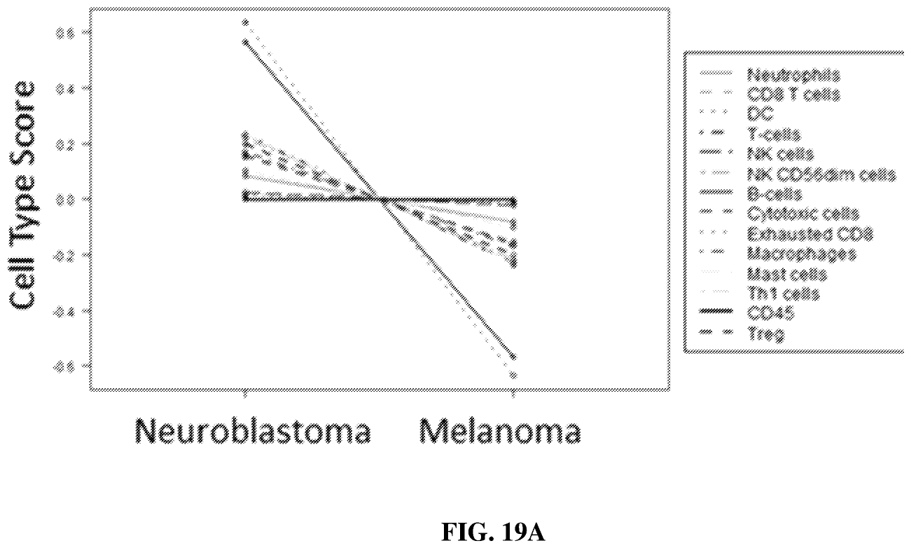

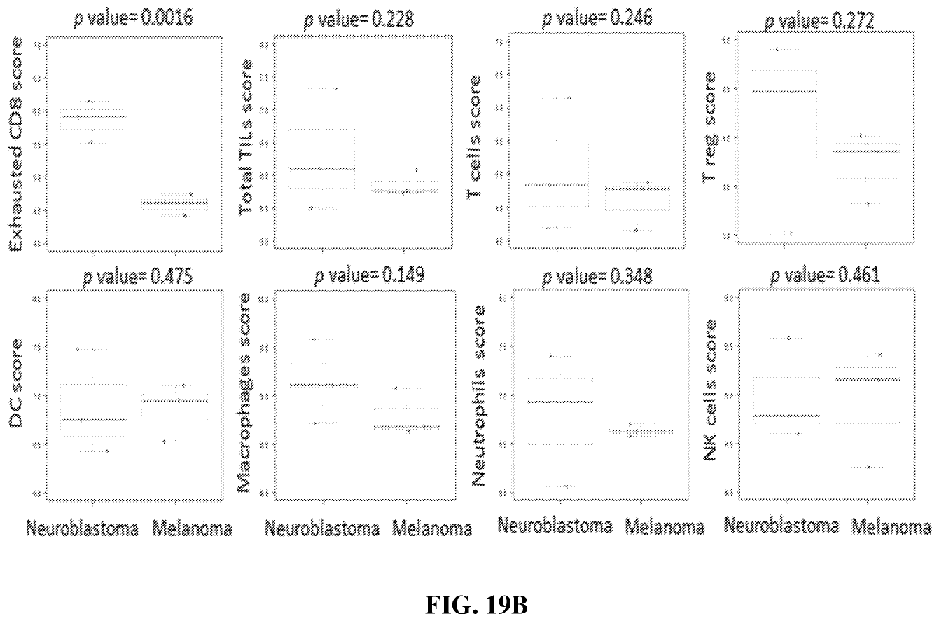

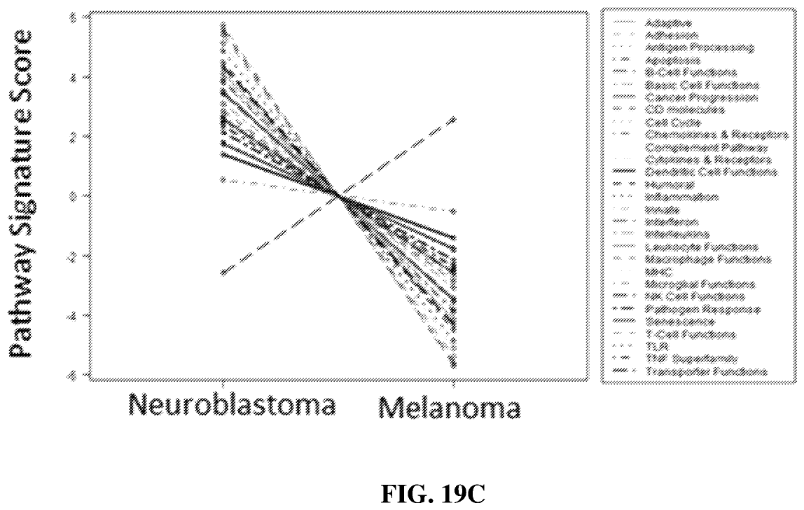

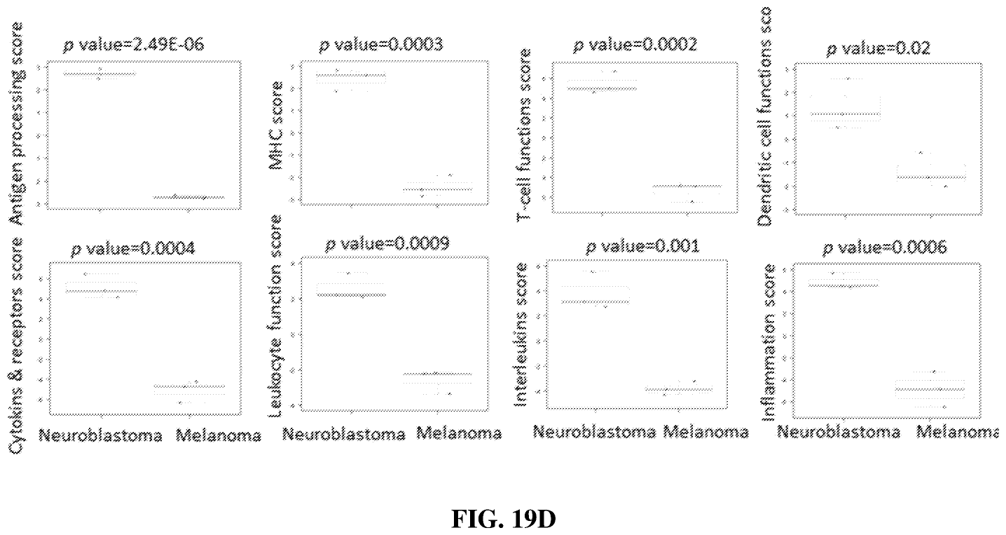

Prior work indicates that whole cell vaccination combined with immune checkpoint inhibitors (ICI) is remarkably effective for eradicating a high dose (2×10⁶ cells) tumor challenge in a Neuro2a neuroblastoma model, but cure in the low dose (1×10⁴ cells) inoculation of B16-F10 melanoma is rare. To evaluate differences in immune sensitivity of the mouse neuroblastoma model to tumor vaccination, established untreated tumors were immune-phenotyped with Nanostring nsolver Pancancer immune profiling platform and compared them to similar sized tumors in the melanoma mouse model. Surprisingly, immune cell infiltrates were very similar in both groups, however a clear and significant difference was observed in the greater number of exhausted CD8 T-cells infiltrating the neuroblastoma tumors (FIGS. 19A and 19B). An evaluation of immune pathways in the tumor environment revealed a substantial increase in activation of these pathways in the neuroblastoma tumors suggesting functional immunity albeit inadequate to completely control tumor growth with high dose tumor inoculation (FIGS. 19C and 19D). Together, the inactivation of immune pathways and the absence of post-activated, exhausted T-cells in the melanoma tumors, suggests that infiltrating immune cells remain in a paralyzed state and are unable to mount significant tumor immunity. This observation re-affirms the finding that checkpoint inhibitors alone (CTLA-4 and PD-L1 blocking antibodies) without vaccination enable enhanced immunity in the neuroblastoma model but fail to impact tumor immunity in the melanoma model. These findings predict that different pathways of immune-suppression are engaged in the melanoma compared to the neuroblastoma tumors and the mere presence of immune cells (immune-rich or T-cell inflamed) in the tumor micro-environment does not predict effective tumor immunity.

ApoE is Highly Expressed in the Melanoma B16-F10 Cell Lines and apoE Serum Levels Rise with Tumor Growth In Vivo

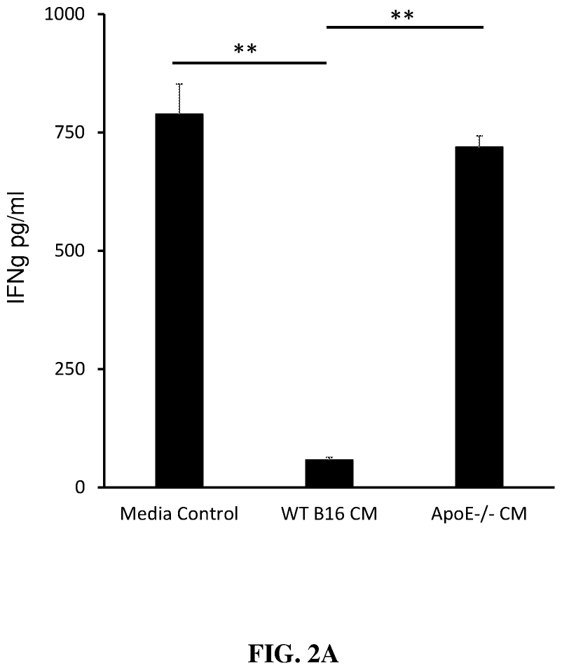

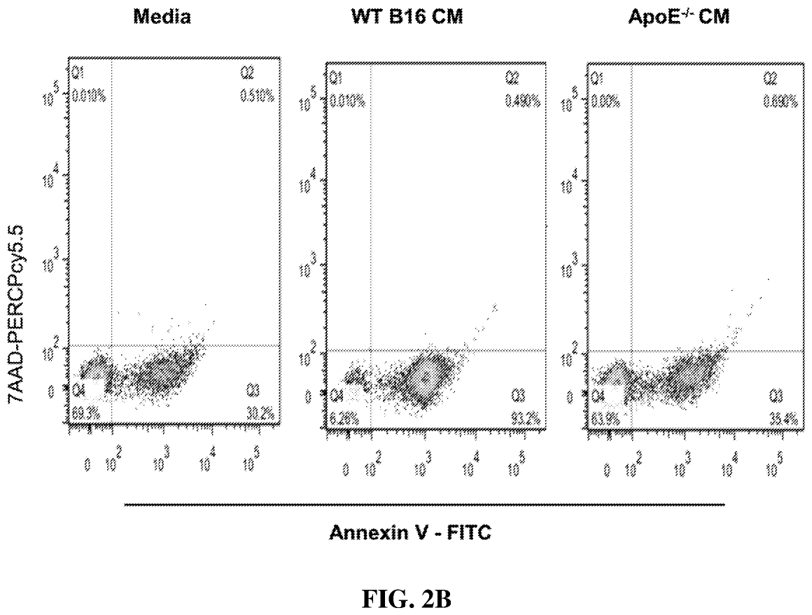

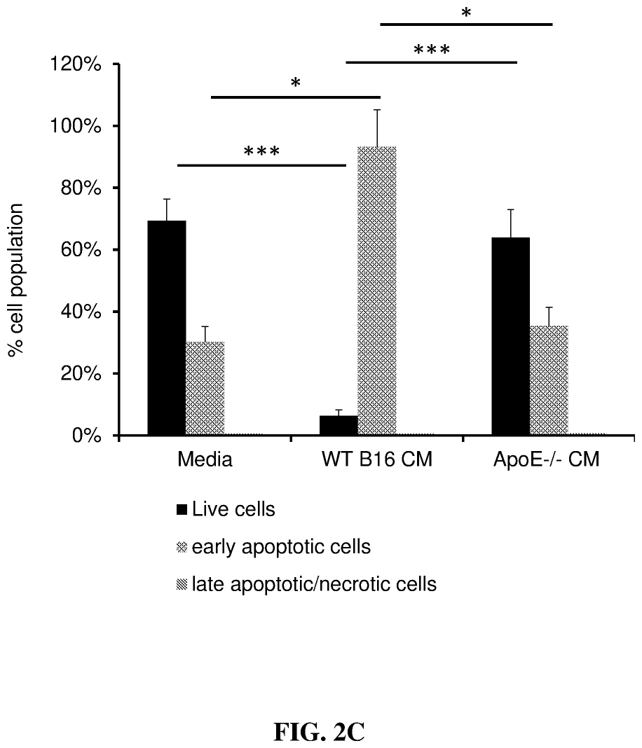

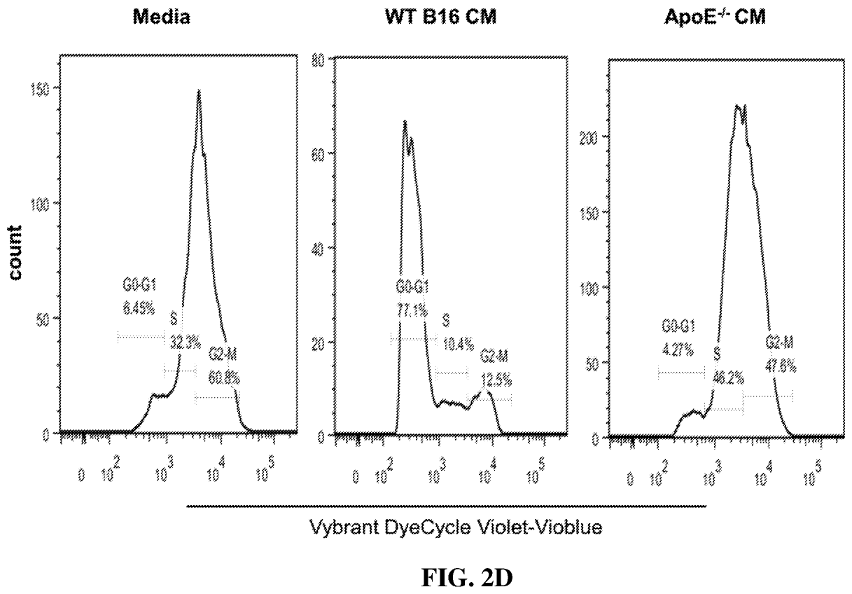

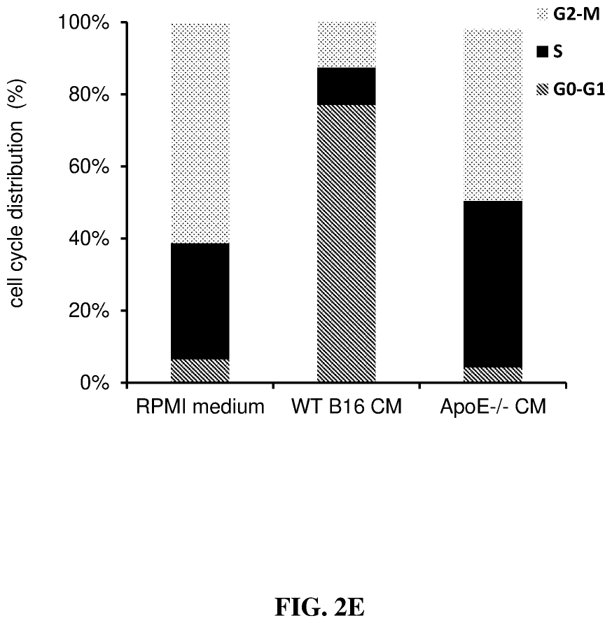

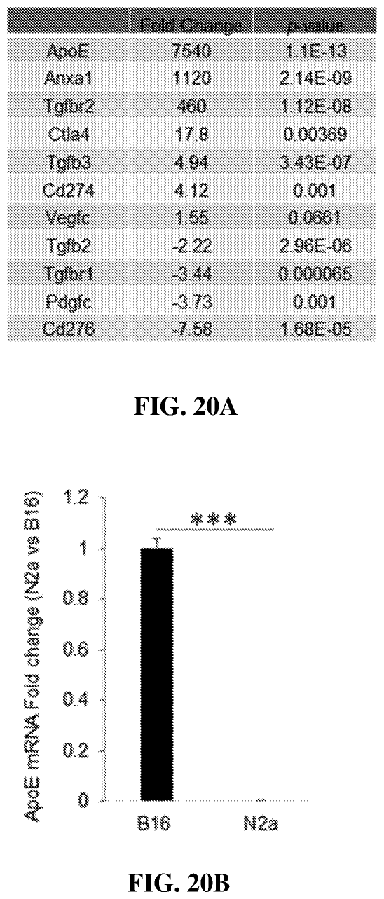

To understand immune differences between the cell lines, we compared immune gene profiles using the Nanostring nsolver Pancancer immune profiling panels. There were remarkable similarities among the 752 genes evaluated between Neuro2a and B16-F10 cell lines, but several differences in immune suppressor genes stood out (FIGS. 2A-2B). Most remarkably, apoE mRNA is seven thousand five hundred and forty-fold greater in the B16 cell line than in Neuro2a cells (FIG. 20A). This result was further validated by real time qPCR (FIG. 20B).

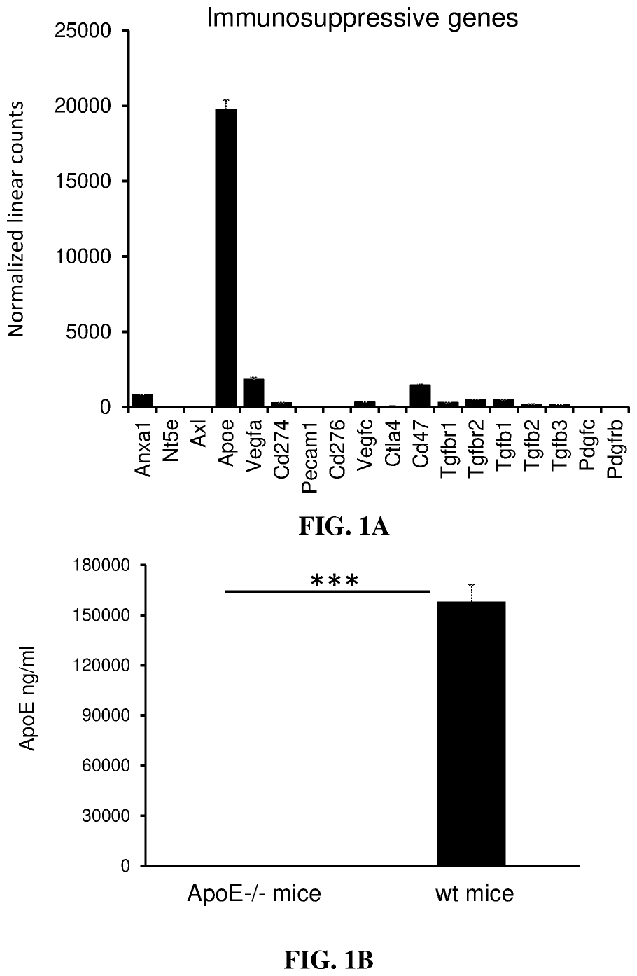

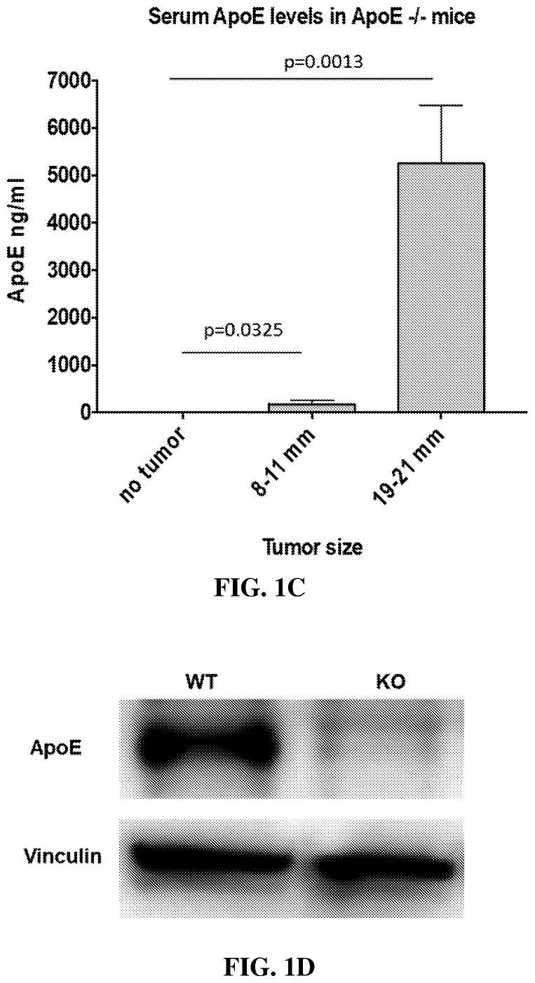

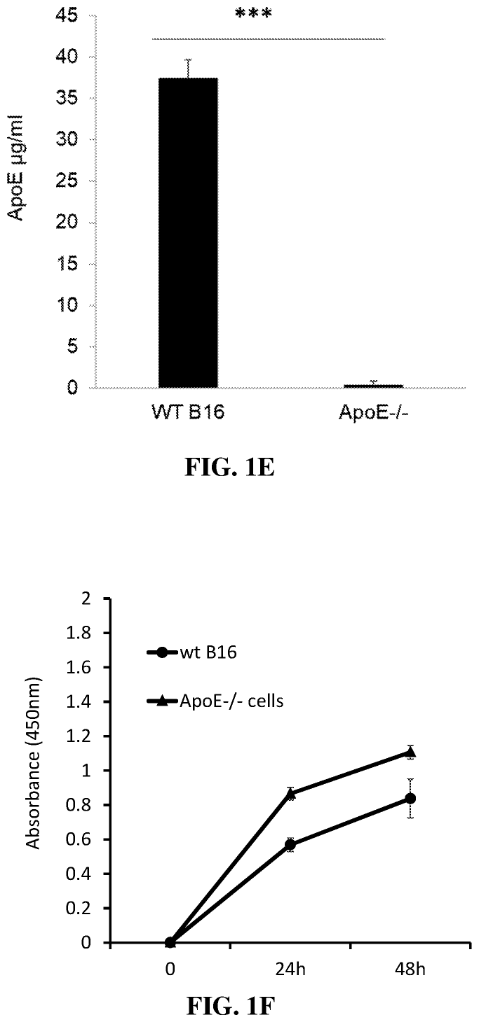

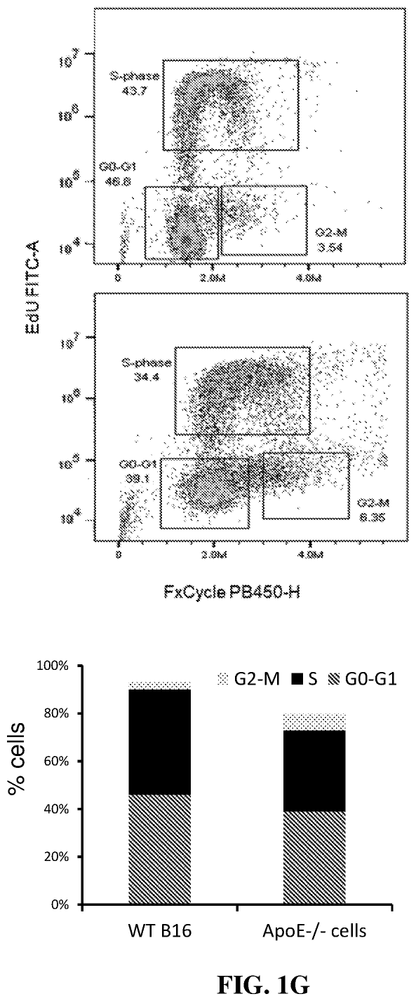

The presence of multiple genes associated with immunosuppression in the B16-F10 melanoma cell line, ApoE was the most highly expressed immune-suppressive transcript in the cell line. (FIG. 1A). ApoE is constitutively expressed in WT C57/BL6 mice at high levels (FIG. 1B). To evaluate the systemic levels secreted from the mouse melanoma tumor itself, we monitored the level of serum apoE in apoE KO (apoE^{-/-}) mice inoculated with 10⁴ WT B16 (F10) cells subcutaneously (s.c.) injected in the right thigh. Blood and tumors were collected at various sizes until the tumors reached a max of 21 mm in any dimension. The serum levels of apoE in these tumor-baring apoE^{-/-} mice increased progressively and considerably with tumor growth (FIG. 1C).

### ApoE Enables Tumor Growth and Protects Against Tumor Immunity

To further investigate the function of apoE in tumors, we generated apoE^{-/-} B16 cell lines with CRISPR-Cas9 gene deletion. To confirm suppression or deletion of apoE protein, we performed western blot analysis on total protein lysates from B16 WT and apoE^{-/-} single clones. The data shows diminished apoE expression in the apoE^{-/-} single clones (FIG. 1D). The apoE protein secreted from each individual clone into culture media was quantified by ELISA assay and correlated with the protein expression pattern of western blot analysis confirming KO of apoE in the cell lines. (FIG. 1E)

We then evaluated the in vitro proliferation rate (FIG. 1F) and cell cycling (FIG. 1G) of the WT and KO cell lines to determine if targeting apoE had an effect on cell viability and proliferation. We then evaluated the in vitro proliferation rate (FIG. 1F) and cell cycling (FIG. 1G) of the WT and KO cell lines to determine if targeting apoE influenced cell viability and proliferation. There is no statistically significant difference between WT and ApoE−/− cells in their proliferation rate or cell cycles (FIGS. 1F-1G).

### Example 3: ApoE Secreted into Media from B16 Melanoma Tumor Cells Inhibits T-Cell Function

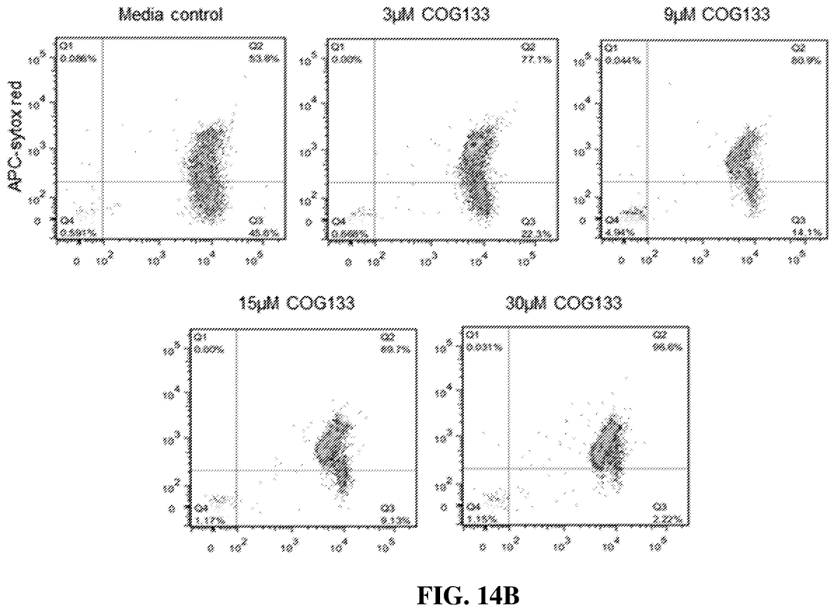

To investigate the effect of tumor cell secreted apoE on activated T cells, splenic derived C57/BL6 T cells were cultured in control media and melanoma B16 WT or apoE-1-conditioned media (CM) in which the T cells were activated with CD3/CD28 beads for 48 hr. Cytokine secretion, apoptosis and proliferation of the cultured mouse T cells were examined and compared to T cells cultured in the RPMI media that served as controls (FIGS. 2A-2E). IFNγ production (FIG. 2A) and T cell viability (FIGS. 2B and 2C) were significantly (P<0.05) suppressed when T cells were cultured in WT B16 CM at 48 hr of incubation as compared to the RPMI media control, whereas apoE^{-/-} conditioned media was similar to RPMI control media alone and did not inhibit T cell activity nor viability (FIGS. 2A, 2B and 2C). In addition, cell cycle distribution showed that WT conditioned media arrested activated T cells in the G0/G1 phase while apoE^{-/-} conditioned media-maintained T-cells in the S and G2M phase of the cell cycle, similar to control media without prior exposure to tumor cells. (FIGS. 2D and 2E). These results indicate that conditioned media derived from B16 tumor cells induces arrest of stimulated T cells in the G0/G1 phase of the cell cycle, resulting in apoptosis and suppression of cytokine production. In contrast, the singular absence of apoE in conditioned media rescues the activated T cell phenotype when stimulated with CD3/CD28 beads. To further define the cytokine response, a ProcartaPlex multiplex immunoassay was used and multiple cytokines/chemokines were quantified in the stimulated T-cell media from the same experiment. Ten out of 27 detectable cytokines suppressed by WT conditioned media returned to baseline levels or were upregulated when T cells were cultured in apoE^{-/-} CM. These included effectors: IFNγ, IFNα, TNFα; stimulator, IL18; inflammatory factor: IL4, IL13; chemo attractive factor: MCP-1, MCP3, MIP-1α, MIP-10; and regulatory factor: IL-10 (P<0.05) cytokines. IL6, CCL-5 (Rantes) and GroaKC were downregulated (FIG. 14A).

To further delineate the T-cell suppressive effect directly, COG133, a fragment of apoE peptide, which competes with the apoE holoprotein for binding the low-density lipoprotein (LDL) receptors and acts as an apoE mimetic, was tested to determine its effect on T cell activation. T cells were activated with CD3/CD28 dynabeads and the effects of COG133 (0, 3, 9, 15 and 30 μM concentrations) on T cell apoptosis were evaluated. The T cell viability was diminished in a dose dependent manner (FIG. 14B). These results confirm the immunomodulatory role of apoE on activation of T cells showing robust and extensive suppression of T cell function.

### Example 4: ApoE Secreted from B16 Melanoma Tumor Cells in Culture May Also Impair Activation of Pro-Inflammatory Dendritic Cells

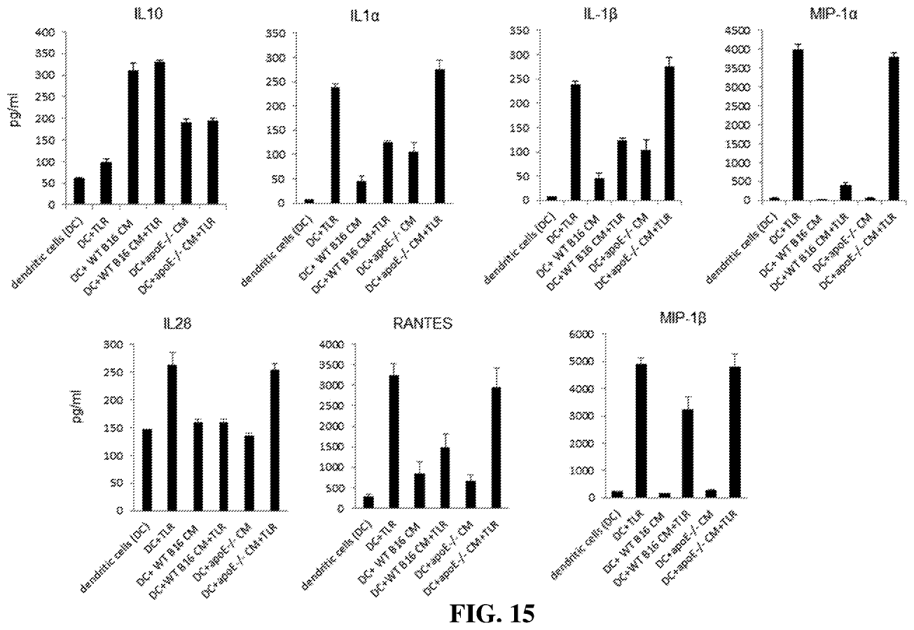

Activation of innate antigen presenting cells like dendritic cells (DC) are critical for effective induction of immunity. The effect of conditioned media on toll like receptor (TLR7/8) stimulated primary bone marrow derived dendritic cell activation was tested. B16 WT conditioned media (CM) suppressed DC activation as determined by cytokine production, but this effect was absent in in media from apoE^{-/-} B16-F10 cells (FIG. 15). Multiplex results showed that secretion of the suppressive cytokine IL10 is diminished when DC were cultured in apoE^{-/-} CM compared to culture in WT CM. In a pro-inflammatory fashion IL1α, IL1β, RANTES, MIP1α, MIP1β, IL28 are all increased with apoE^{-/-} CM compared to WT CM (FIG. 15).

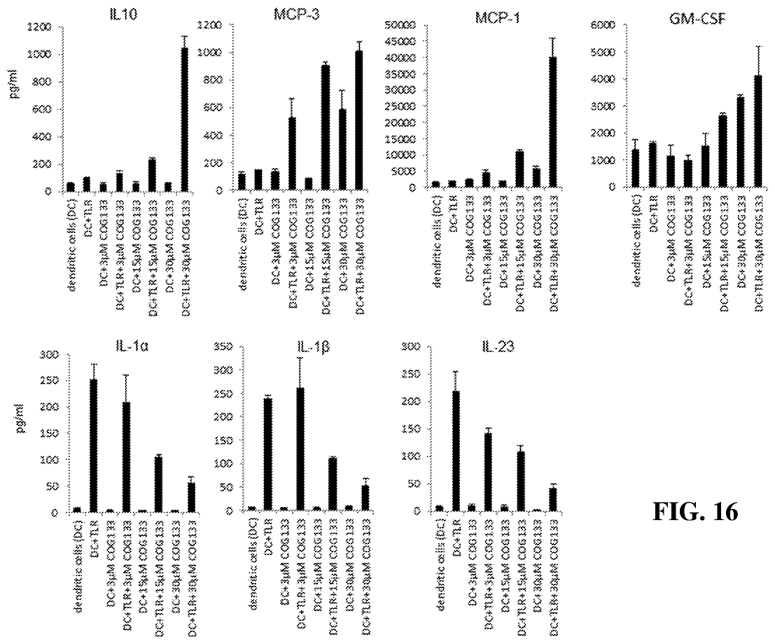

To further delineate the DC suppressive effect directly, the effect of the apoE peptide mimetic, COG133 on DC activation was tested. In a dose dependent fashion, COG 133 induced secretion of the anti-inflammatory cytokine, IL-10 from activated DC. Furthermore, COG133 suppressed IL-1α, IL-1β and IL-23 in a dose dependent manner suggesting the immune-modulatory role of ApoE on DC function (FIG. 16).

### Example 5: ApoE Peptide Mimetic COG133 Inhibits Cytokine Secretion Induced by Immunogenic Tumor Cells, while Anti-APOE Blocking Antibody Enhances Cytokine Secretion in Tumor Cell/Splenocyte Reactions

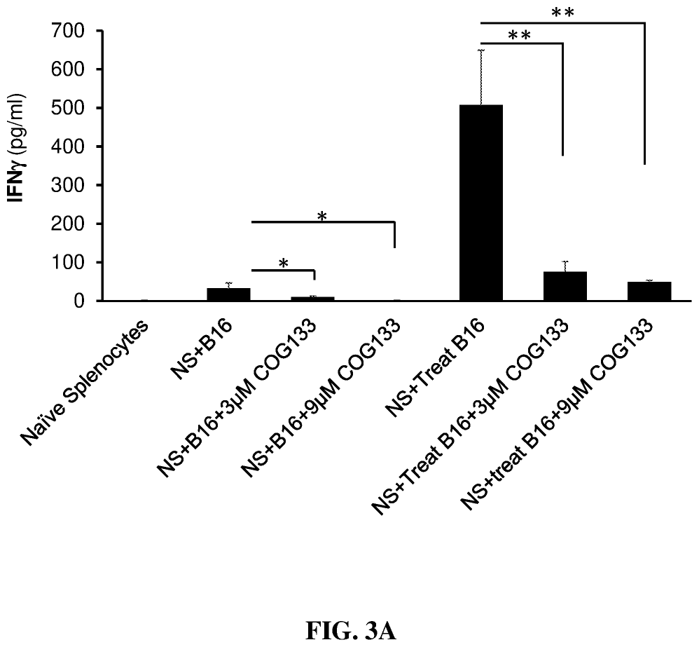

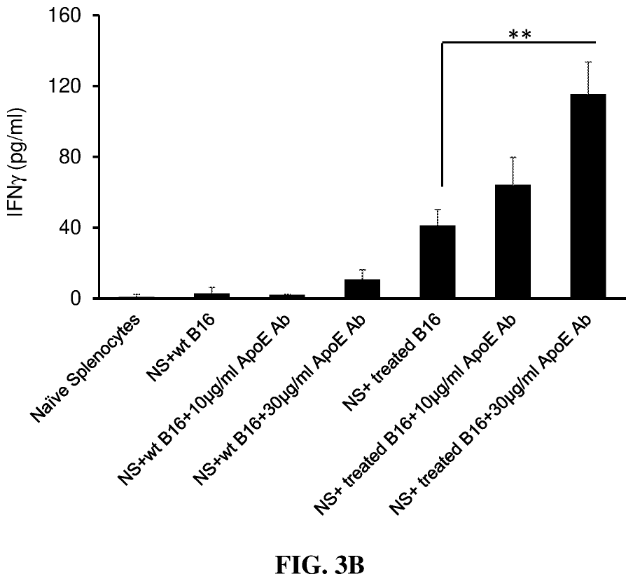

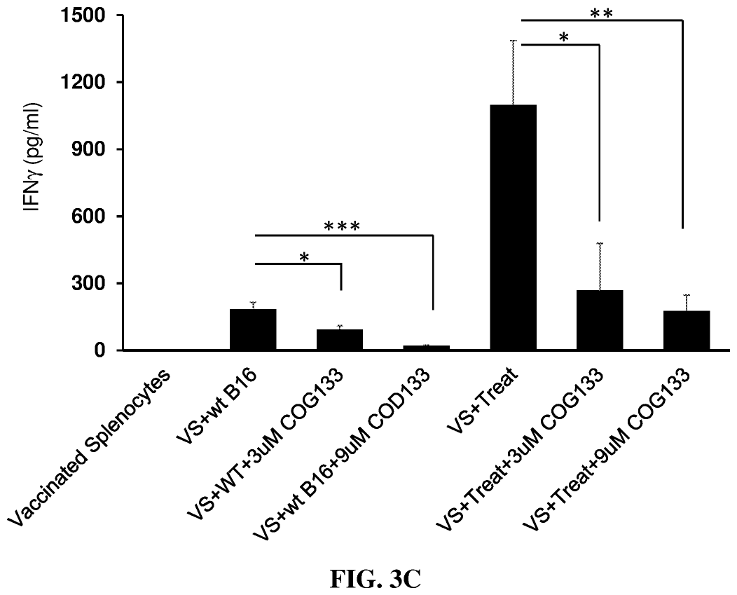

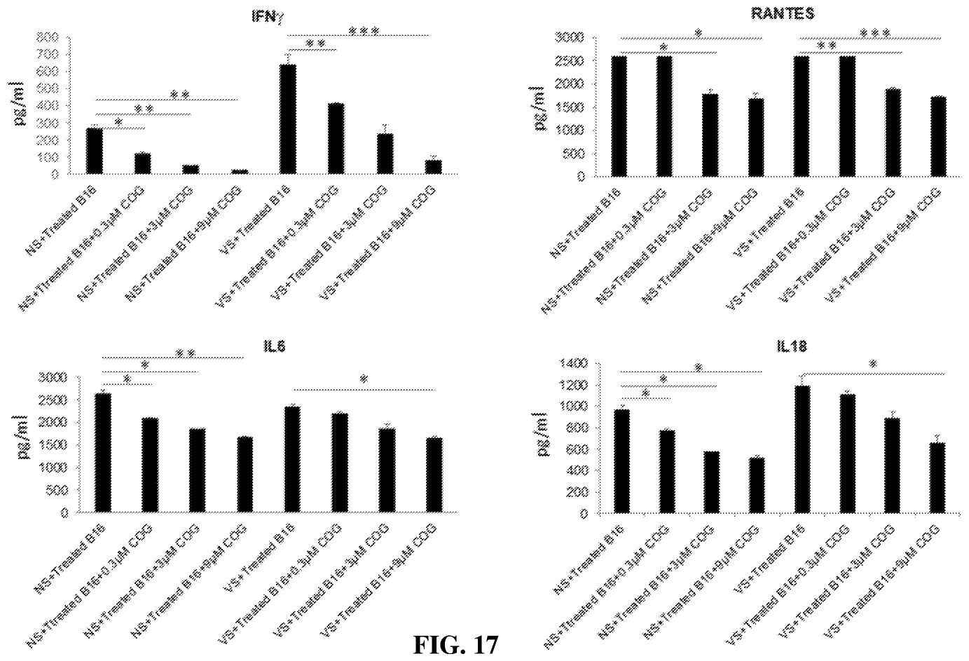

To further investigate the influence of tumor secreted apoE on immune cell function, immunogenic B16 tumor cells (B16 cells treated with Myc inhibitors (0.25 μM BET+0.25 μM JQ1 for 4 days) were irradiated at 60 Gray and co-cultured with naïve C57/BL6 splenocytes in the presence of either apoE mimetic COG133 at 0.3, 3 and 9 μM or anti-APOE blocking antibody at 1, 10 and 30 μg/ml concentrations. Prior work has shown the immunogenic effect of treating cancer cells with Myc inhibitors and irradiation. IFNγ production was quantified by ELISA at 48 h. Splenocytes produced high levels of IFNγ when co-cultured with Myc-inhibited immunogenic B16 cells as shown in FIGS. 3A-3C. Exposure of these cells to apoE mimetic COG133 repressed IFNγ production (6-fold reduction) (FIG. 3A), while the presence of Anti-APOE antibody enhanced IFNγ production (3-fold increase) (FIG. 3B). We also tested IFNγ production from vaccinated splenocytes following co-culture with treated and untreated B16 cells in the presence of COG 133. To obtain vaccinated splenocytes, 10\hat{ }4 WT B16 tumor cells and 100 μg/ml anti-CTLA4 antibody were administered to C57BL/6 mice on day 0, and splenocytes were collected at day 7 after tumor cell inoculation. Compared with naïve splenocytes, vaccinated splenocytes produced dramatically greater level of IFNγ, especially when they were co-cultured with Myc inhibited B16 tumor cells. This high level IFNγ was also suppressed by COG 133 at a dose dependent manor (FIG. 3C). In addition to IFNγ, we also quantified other cytokine/chemokines using ProcartaPlex multiplex immunoassay. Fourteen out of 23 detectable cytokines were significantly upregulated when splenocytes were co-cultured with Myc-inhibited B16 tumor cells, including effectors: IFNγ, TNFα; stimulators, IL18, G-CSF, M-CSF; inflammatory factor: IL6; chemo attractive factors: CCL-5 (Rantes), CXCL-1, CCL-2, CCL-7, CXCL-2, CCL3; and regulatory factors: IL-10, IL6 (P<0.05). Within these 14 cytokines, four of them including IFN□, IL6, IL18 and RANTES (CCL5) were suppressed after exposure to apoE peptide mimetic COG133 in a dose dependent manner (FIG. 17).

### Example 6: The apoE Receptors lrp8 and ldlr are Dominantly Expressed on Activated T Cells and Dendritic Cells and Blocking Lrp8 Enhanced T-Cell Activation In Vitro

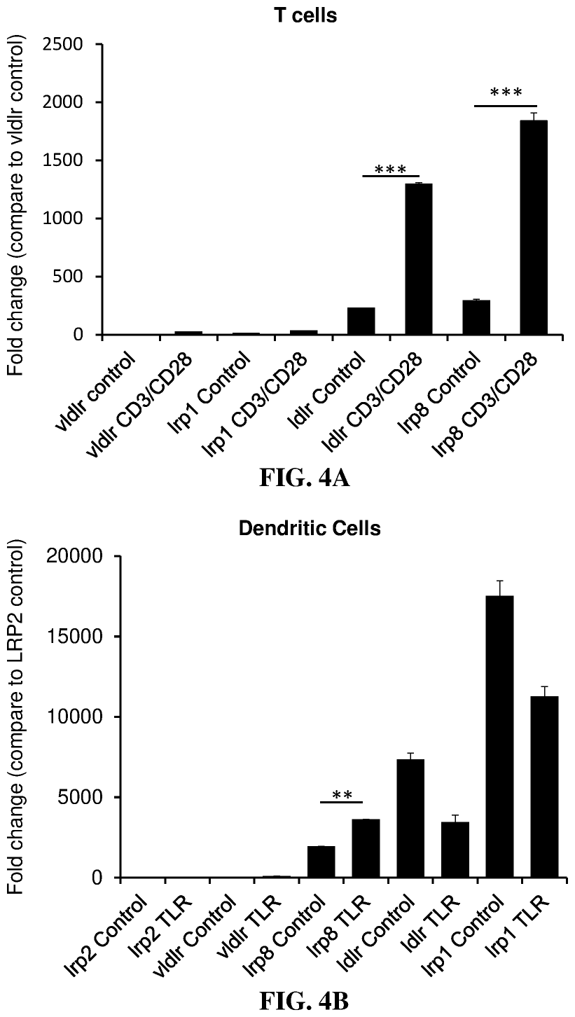

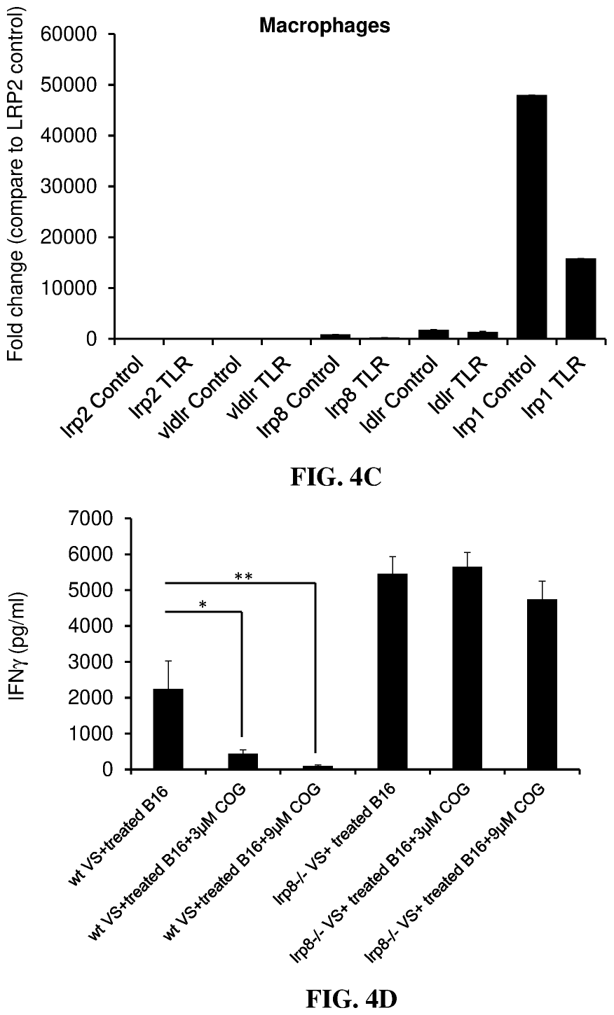

The pattern of expression of apoE receptors on T cells and dendritic cells is not fully characterized. Here the expression of apoE receptors on T cells, dendritic cells (DC) and macrophages was examined using qPCR. All five of the receptor transcripts were shown to be expressed on T cells, DCs and macrophages. Ldlr and lrp8 were dominantly expressed on T cells (FIG. 4A) whereas lrp1, lrp8 and ldlr are highly expressed on DC (FIG. 4B). Lrp1 is highly expressed in macrophages (4C). Vldlr expression was relatively low, and lrp2 was barely detectable on these three cell types. The effect on expression of these receptors by stimulating T cells with CD3/CD28 beads and stimulating DCs and macrophages with a TLR7/8 agonist was further examined. Expression of lrp1, lrp8, ldlr and vldlr were all significantly upregulated on T cells following activation (FIG. 4A), whereas only lrp2 and lrp8 were increased on DC after TLR stimulation (FIG. 4B). This data together with previous studies suggest that ldlr and lrp8 (apoER2) are dominantly expressed and may be prominently engaged in apoE-mediated immune cell suppression.

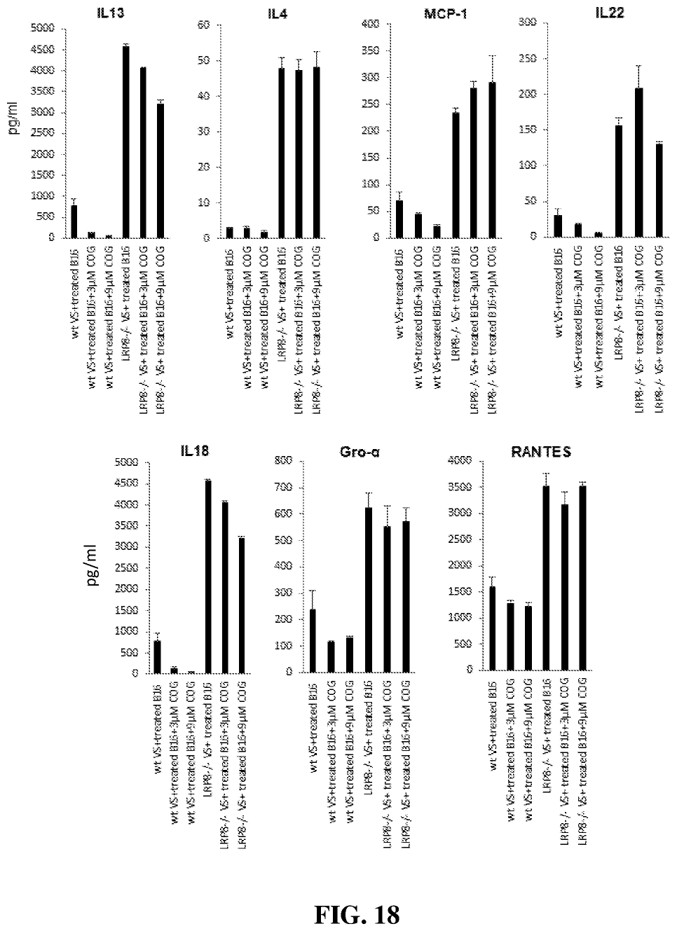

To functionally define the role of the lrp8 receptor engagement in apoE suppression, vaccinated splenocytes from lrp8^{-/-} mice were isolated. These cells were co-cultured with BET/JQ1 treated (Myc-suppressed) immunogenic B16 cells for 48 hr with or without exposure to the apoE mimetic COG133. ELISA results show that IFNγ production from splenocytes was inhibited by COG133 in WT mice, however the inhibitory effect was lacking in splenocytes from the lrp8^{-/-} mice (FIG. 4D). In addition, using ProcartaPlex multiplex immunoassay, we also quantified other cytokines/chemokines produced in the reaction. Seventeen out of 27 detectable cytokines showed a similar pattern to IFNγ inhibition in WT mice that was reversed in lrp8^{-/-} splenocytes. These included effector function: IFNγ; stimulator function, IL18, GM-CSF, G-CSF; inflammatory cytokines: IL2, IL3, IL4, IL5, IL9, IL13, IL23, IL12p70; chemo attractant factors: MCP-1, MCP3, MIP-1α, CCL-5 (Rantes); and regulatory factors: IL6, IL-10 (P<0.05). Representative data are shown in FIG. 18. These findings suggest that T-cell function is at least partially inhibited by apoE through the lrp8 receptor pathway.

### Example 7: Targeting apoE Suppresses Tumor Growth with Enhanced Mouse Survival in a Murine Melanoma Model

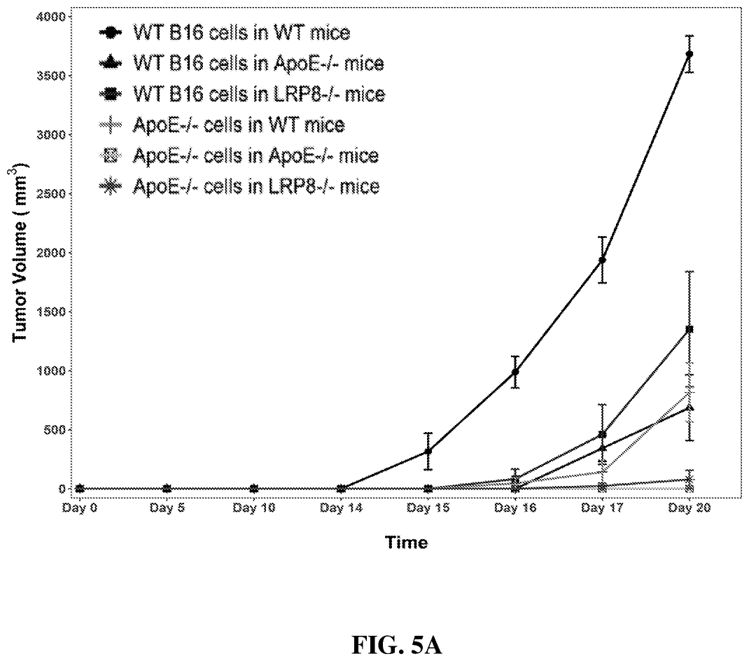

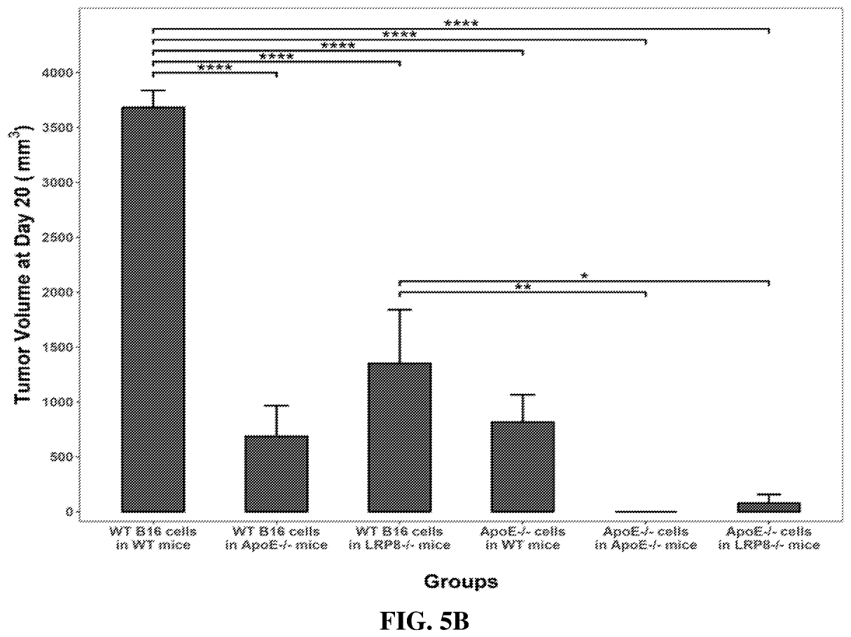

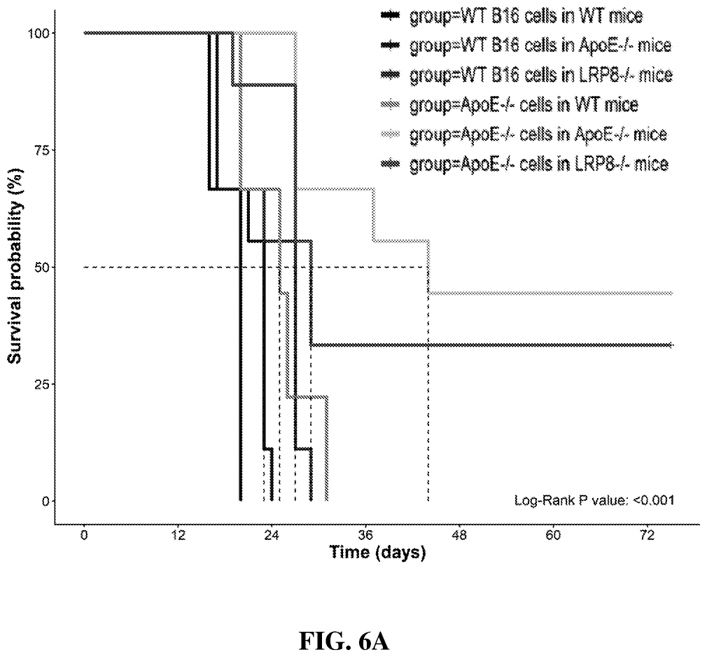

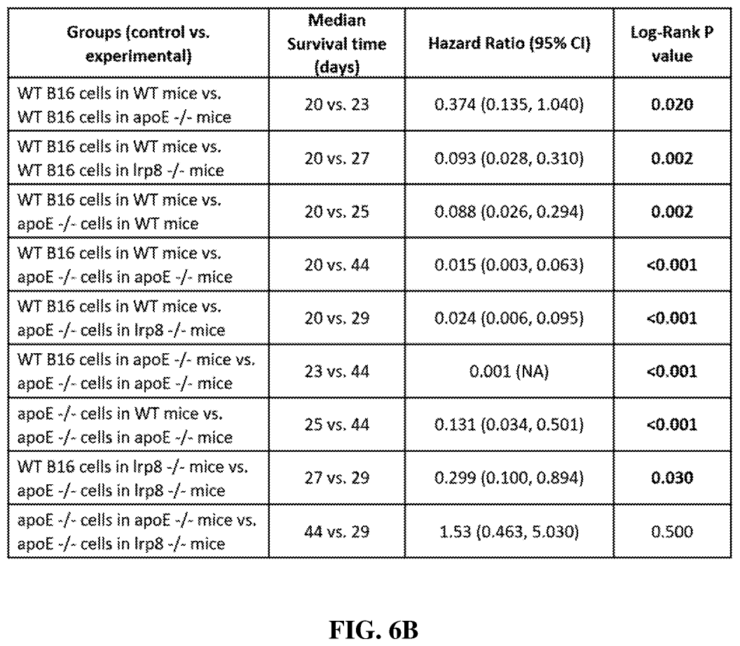

To additionally investigate the role of apoE on melanoma tumor growth in vivo, 1×10⁴ WT or apoE^{-/-} B16 cells were injected into the right flank of WT, apoE^{-/-} and lrp8^{-/-} mice (n=9). The mice from each group were monitored for tumor growth and survival. The results show that targeting apoE in both the tumor cells and the host (apoE^{-/-} B16 cells injected into apoE^{-/-} C57/BL6 mice) results in delayed tumor growth (FIG. 5), and significant rejection of tumor cell inoculation with improved overall survival (FIG. 6). Tumor growth was also impaired when apoE^{-/-} B16 cells were injected into Irp8^{-/-} mice, suggesting apoE/LRP8 receptor engagement is important in mediating the apoE protective effect on tumor growth. Complete deletion of apoE in the tumor cells and in the host was most effective for inhibition of tumor growth, while apoE^{-/-} B16 cells injected into WT mice, or WT B16 cells injected into apoE^{-/-} mice or lrp8^{-/-} mice partially suppressed tumor growth compared to control. The suppressive effect observed on tumor growth in vivo with apoE suppression appears indirect as apoE KO cells proliferate normally.

### Example 8: The Combination of apoE^{-/-} Tumor Cells Administered to apoE^{-/-} Mice Resulted in the Most Profound Activation of Immune Pathway Signaling and Cell Infiltrates in the Tumor Microenvironment

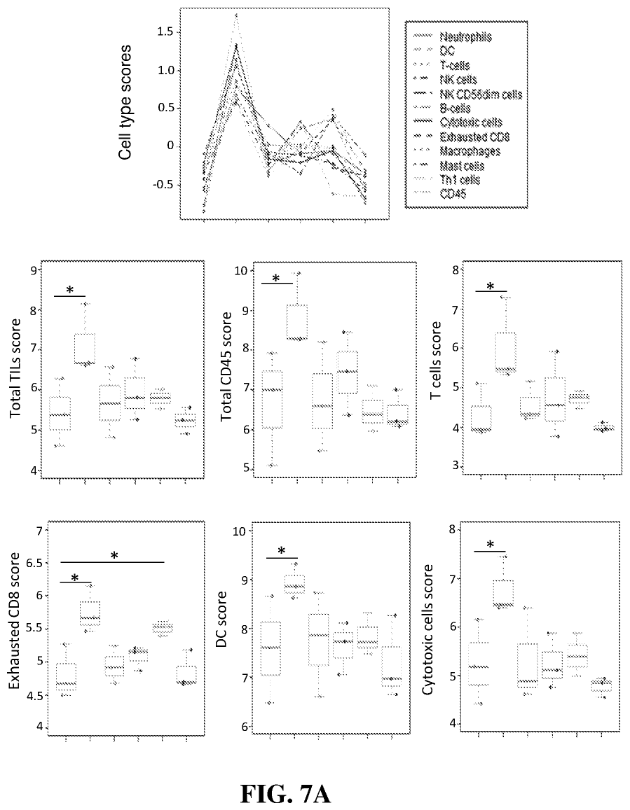

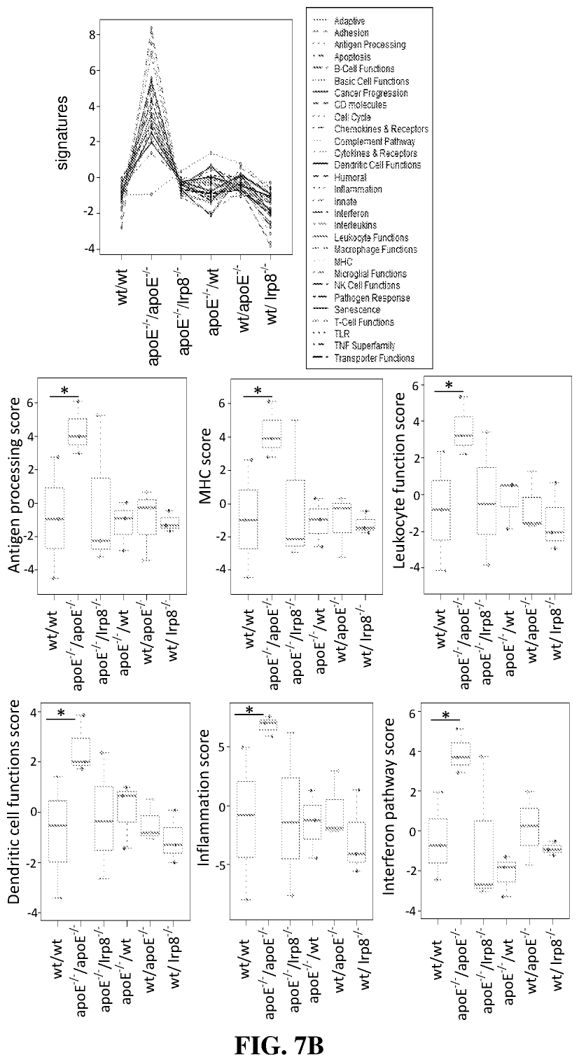

To evaluate if the protective effect against tumor growth with apoE targeting is immune mediated, the first 3 mice from each group that grew tumors to 15 mm were harvested for tumor immune profiling. Nanostring analysis of immune cell infiltrates and activation of immune signaling pathways revealed that the apoE^{-/-} mice in which apoE^{-/-} cells were inoculated, demonstrated the greatest number of immune cell infiltrates (FIG. 7A) as well as the highest activation of immune signaling pathways (FIG. 7B) based on the expression of signature marker gene transcripts when compared to the other groups. Wild type tumors in apoE^{-/-} mice or WT mice receiving the apoE^{-/-} cells demonstrated more activation of immune pathways and cellular infiltrates as determined by RNA transcripts than WT controls, but these effects were only partial and inferior to the immunity induced when apoE was abolished in the system (apoE^{-/-} cells in apoE^{-/-} B57/BL6 mice).

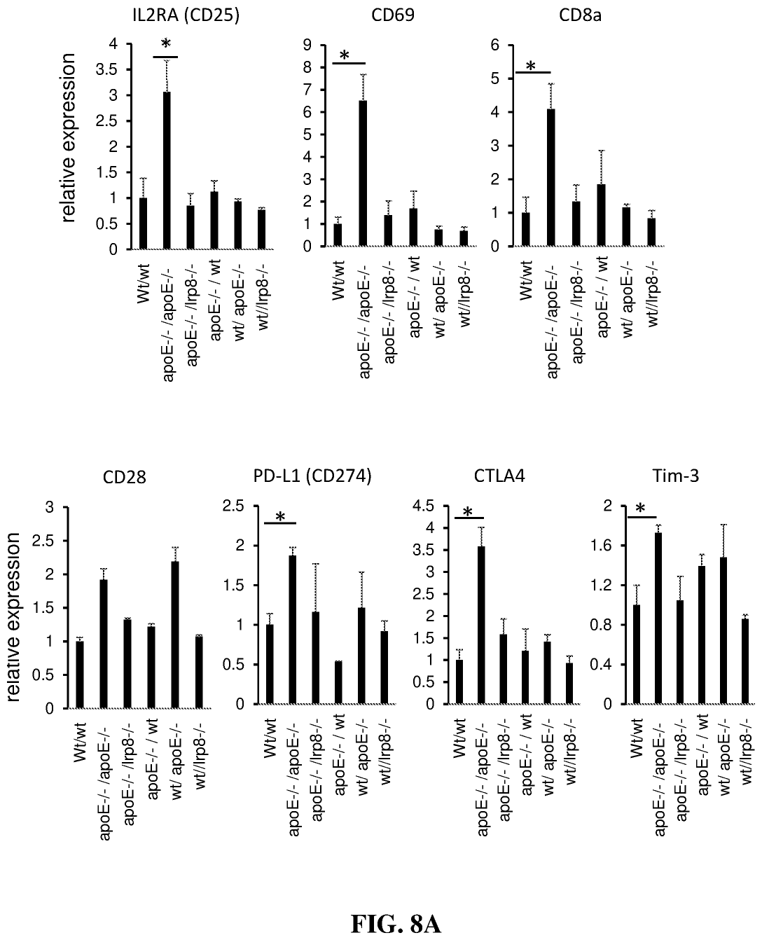

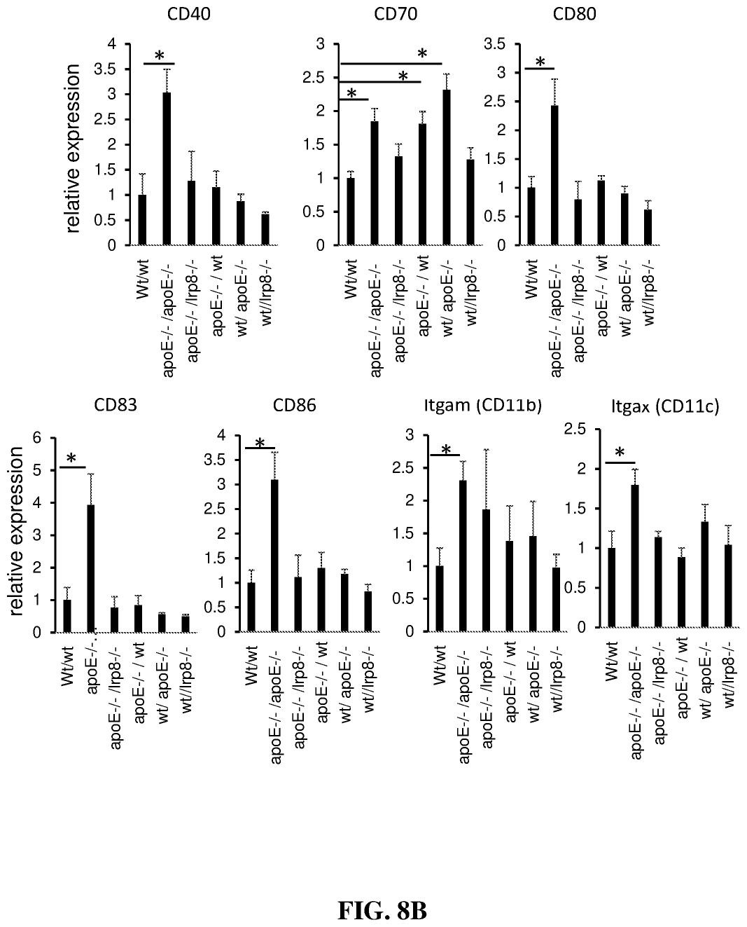

The expression level of the gene transcripts of multiple activation markers for T cells (FIG. 8A) and dendritic cells (DCs) (FIG. 8B) were also compared within 6 tumor groups. Results showed that the markers for activation T cells including interleukin-2 receptor alpha chain (IL2RA, or CD25), CD69, CD8a, CD28, check point inhibitors PD-L1 and CTLA4, and T cell exhaustion marker Tim-3 were all significantly enhanced in the tumor from the apoE^{-/-} mice in which apoE^{-/-} cells were inoculated (apoE^{-/-}/apoE^{-/-}) compared with control group (wt/wt), PD1 and Lag 3 were slightly increased, but they were not statistically significant (data not shown). CD28 was also upregulated in the tumor from the apoE^{-/-} mice in which wt tumor cells were inoculated (wt/apoE^{-/-}) (FIG. 8A). For DCs, the activation genes including CD40, CD70, CD80, CD83, CD86, CD11b and CD11c were all significantly increased in apoE^{-/-}/apoE^{-/-} group compared with controls. CD70 was also increased in wt/apoE^{-/-} and apoE^{-/-}/wt groups. (FIG. 8B). These observations show that apoE secreted from the tumor or produced in the host impair immunity and establish the potent role that apoE plays in suppressing tumor immunity in the mouse melanoma model. Surprisingly, there was no upregulation of immune pathway scores nor enhanced immune cells scores in lrp8^{-/-} mice. These observations do not correlate with in vitro findings nor in vivo growth rates, but this may have been specific to the three mice sampled that developed tumors early in this group.

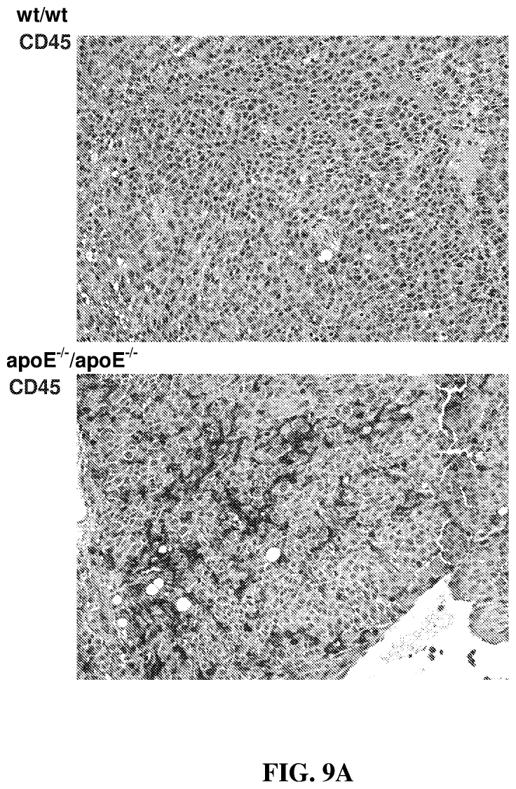

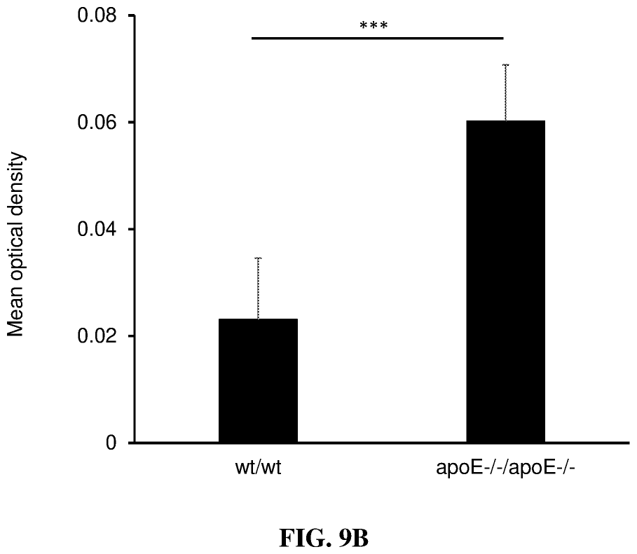

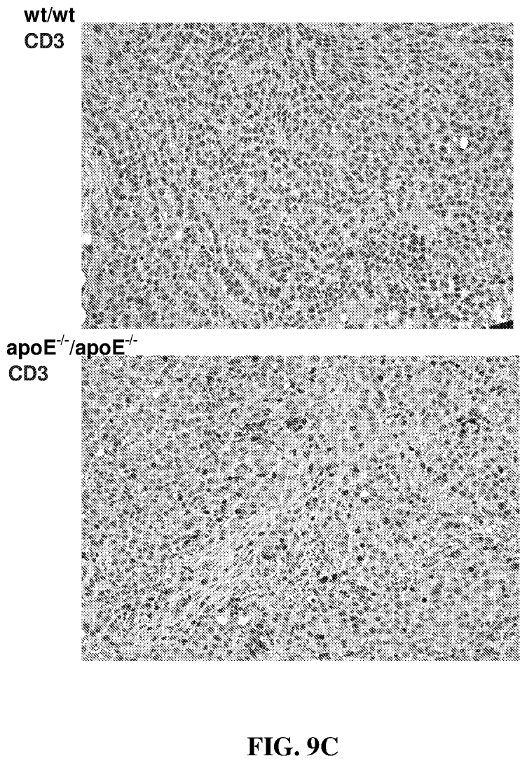

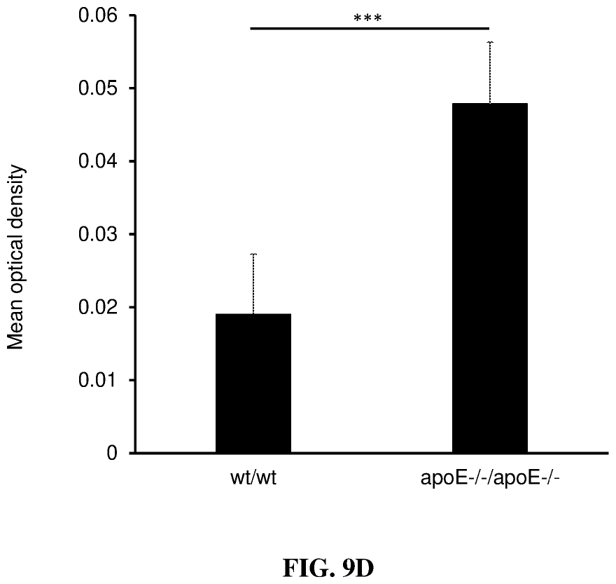

To validate nanostring RNA transcript results in the apoE targeted group, we performed immunohistochemistry staining of the immune cell marker CD45 (lymphocyte common antigen) and CD3 (T cell marker) on the same tumor samples that we used for nanostring analysis. Results showed that the apoE^{-/-} mice in which apoE^{-/-} cells were inoculated, have significantly more CD45 (FIGS. 9A and 9B) and CD3 (FIGS. 9C and 9D) positive immune cell infiltrates than WT control. The other groups were not studied as these gross observations do not have the same objectivity or power of analysis as the nanostring assay.

### Example 9: ApoE Knock Out in B16 Tumor Cells Induces Potent Immunogenicity

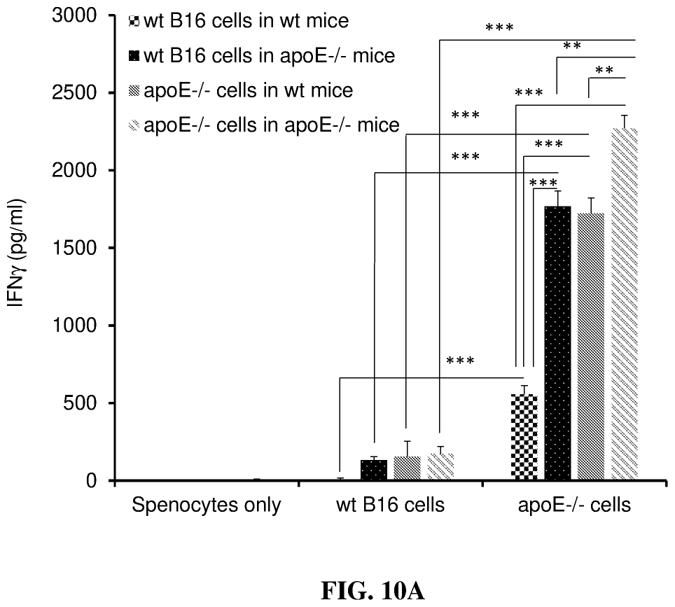

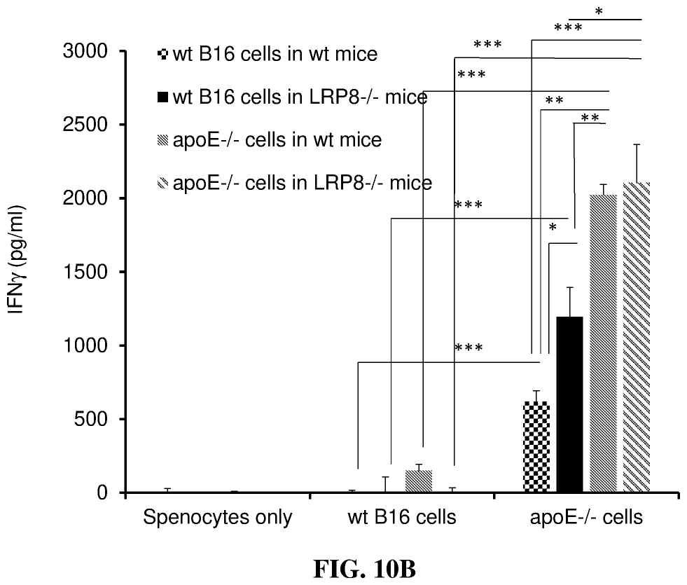

In the mouse model, apoE appears to be an immune modulator critical for enabling tumor cell growth through suppression of immune activation. To determine if knocking out apoE in tumor cells induced immunogenicity, we vaccinated wild type and apoE^{-/-} mice (FIG. 10A) or wild type and lrp8^{-/-} mice (FIG. 10B) with WT B16 or apoE^{-/-} B16 tumor cells and CTLA4 Ab and then collected splenocytes from these vaccinated mice 6 days later. The splenocytes were cocultured with WT B16 or apoE^{-/-} B16 cells to evaluate IFNγ secretion as a marker of induced immunity. ApoE^{-/-} B16 cells induced robust immunity in whatever circumstance they were tested either as the primary immunogen or with re-stimulation of splenocytes (FIGS. 10A and 10B).

These findings re-iterate the potent inhibitory effect of apoE on immune cell activation and present an opportunity to exploit this pathway to enable tumor immunity and cancer immunotherapy.

### Example 10: ApoE RNA-Seq Expression is Abundant in Cutaneous Melanoma but is not Associated with PD1, PD-L1 or Immune Cell Infiltrate RNA-Seq Expression

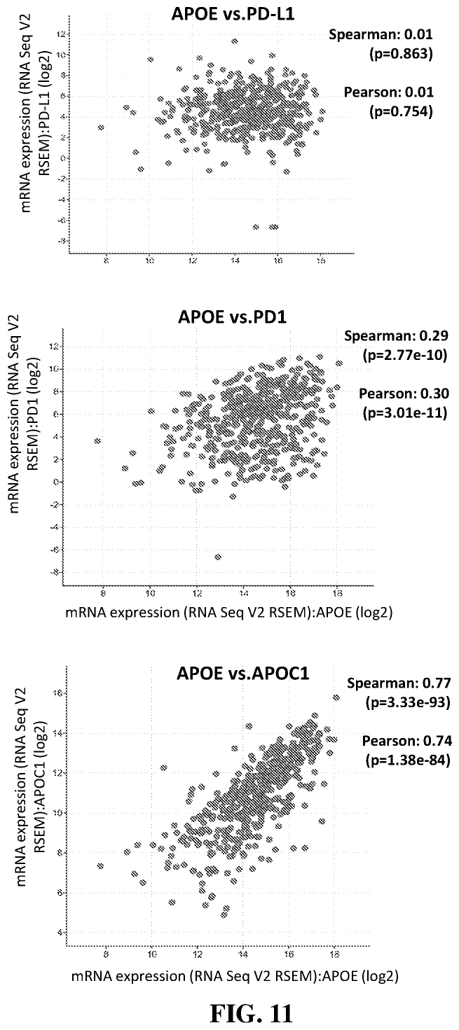

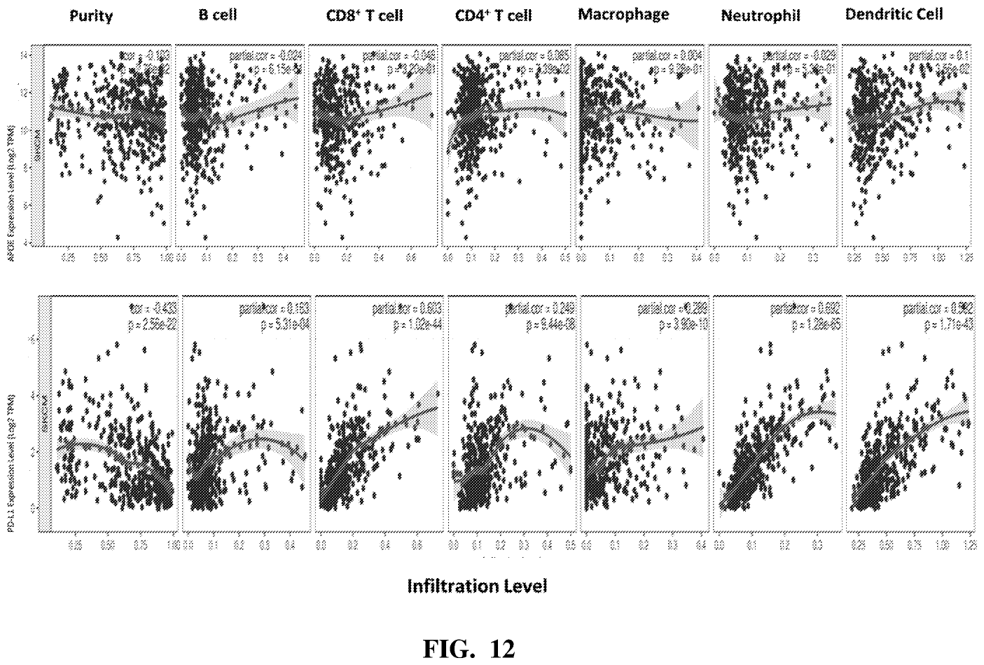

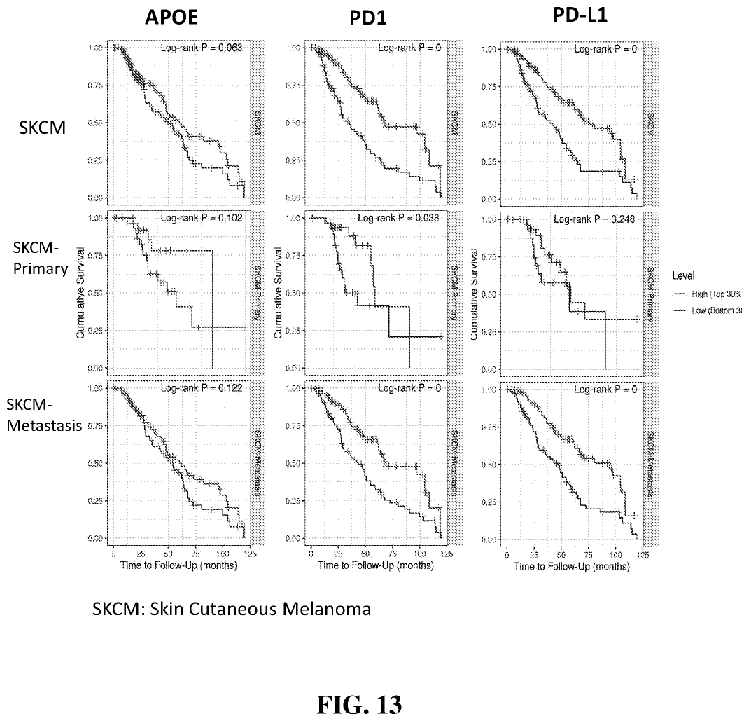

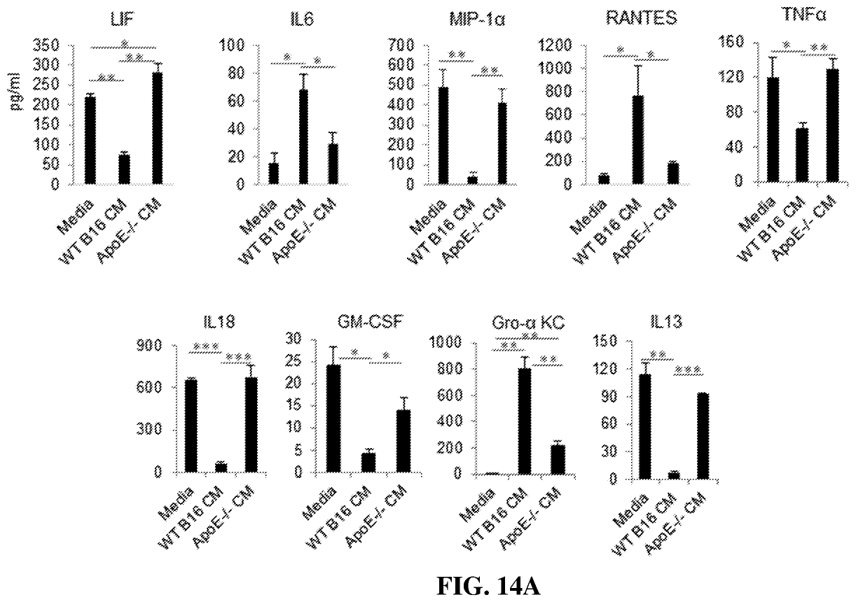

The potent immune-suppressive effects of ApoE in the melanoma mouse model suggests that APOE may be an important regulator of immunity in human melanoma. To evaluate its association with human melanoma, TCGA melanoma datasets based on RNA-seq gene expression values (measured by RSEM algorithm) in 462 patient tumors were analyzed. We evaluated expression and correlation with other checkpoints (FIG. 11), immune cell infiltrates (FIG. 12) and patient survival (FIG. 13). APOE is abundantly present, however, it did not correlate with PD-L1 or PD1 expression, two checkpoints expressed on tumors, but did positively correlate with APOC1 expression APOC1 was used as a positive control in this analysis (FIG. 11). APOE did not correlate with RNA-seq gene expression of T-cell, neutrophil and dendritic cells infiltrates whereas PD-L1 expression correlated with the presence of these three cell phenotypes (FIG. 12). Also, APOE did not correlate with survival at a 30% bifurcate gene analysis, whereas the expression of PD-L1 and PD1 both positively correlated with survival of cutaneous melanoma (FIG. 13).

The high expression of checkpoints like PD-L1 and PD1 associated with cell infiltrates and survival curves, suggest that these genes are expressed in inflammatory tumors that have a better prognosis. APOE expression however appears to be independent of inflammatory phenotype in these tumors and may act as a separate and independent pathway in suppressing anti-tumor T-cell immunity.

### Example 10: Discussion

Distinctly different response to the same tumor vaccine protocol in a mouse neuroblastoma and melanoma tumor model has previously been shown. The neuroblastoma mouse model was remarkably sensitive to tumor vaccination even at a high dose of tumor cell inoculation whereas a low dose melanoma model was surprisingly resistant. This differential response could imply that intrinsic tumor cell characteristics and/or differences in tumor/host immunity are present that may account for differences in immune resistance.

In the current study immunosuppressive modulators in the melanoma model were assessed, and the modulator that was most highly expressed by qPCR in the B16-F10 melanoma is ApoE. Over 700 immune associated gene transcripts in the melanoma B16-F10 cells were profiled, identifying 18 genes with known immunosuppressive characteristics that were detected. ApoE was notably detected and found to be more abundant than any of the other known immunosuppressive transcripts identified. Besides its role in cholesterol transport, Apolipoprotein E (apoE) has considerable immunomodulatory properties. ApoE is shown to suppress lymphocyte proliferation and modulate immune activation by acting on antigen-presenting cells, implicating apoE as a suppressant of immune function. ApoE was found to be abundantly expressed in the B16-F10 melanoma cell line and was actively secreted by these tumor cells into the serum of the host as the tumors establish and grow. These findings raised suspicion that apoE contributes to immune escape.

Both the expression of apoE as well as the secretion of apoE from the tumor cells seems to have clear immunosuppressive effects. Our studies show that tumor cells depleted of apoE can stimulate remarkable immune activation in co-culture experiments. The conditioned media from apoE^{-/-} tumor melanoma cells alone did not suppress T-cell function like that of WT tumor cell conditioned media. To understand the mechanism by which apoE functions we investigated specific effects of the apoE peptide mimetic COG133 on T-cell and DC function. The suppressive effect appears to be evident on both stimulated DCs and T-cells as determined by cytokine secretion. We identified the dominant receptors on these cells showing that of the low-density lipoprotein (ldlr) receptor family evaluated, lrp8 and ldlr were most abundant and responsive to stimulation in T-cells while ldlr and lrp1 were present in DCs. In co-culture experiments, the suppressive effect of apoE on vaccinated splenocytes was absent when the splenocytes were harvested from lrp8^{-/-} mice. These findings suggest that T-cell function is at least partially dependent on apoE-lrp8 receptor engagement.

To investigate the premise of ApoE being a potent immune checkpoint, we challenged WT, apoE^{-/-} and lrp8^{-/-} mice with WT and ApoE knock out B16-F10 melanoma tumor cells. Results show remarkable suppression of tumor growth when ApoE is absent from the system with significant tumor elimination when compared with WT controls. ApoE in tumor cells or endogenously produced in the mice was capable of suppressing tumor immunity and significant tumor immunity only occurred when ApoE was absent from both tumor cells and the murine host. Immuno-phenotyping of the tumor micro-environment in mice that grew tumors, revealed the highest levels of immune pathway activation and immune cell infiltrates in the apoE^{-/-} mice that received apoE^{-/-} B16 melanoma cells as detected by RNA transcripts. The effect of apoE seems to be at least partially mediated by its interaction with the Lrp8 receptor as this group also had slower tumor growth and rejection of tumor cells when apoE^{-/-} melanoma cells were inoculated into Lrp8^{-/-} mice, endorsing the in vitro findings in splenocyte studies. However, the immune mediated effect was not as apparent in the lrp8^{-/-} mice, as activation of immune pathway and cellular RNA transcripts were not universally increased liked that observed in the complete apoE knock out tumor/mouse model. These findings may be limited and underestimate the ultimate tumor immunity as the tumor micro-environment was analyzed from the first three mice in each group that developed tumor and do not necessarily represent the mice with delayed or absent tumor growth.

Several potential shortfalls of our findings are evident considering prior literature. The lack of myeloid-derived suppressor cells (MDSC) analysis in the B16-F10 mice models used is evident. Previous studies have shown that the liver-X nuclear receptor (LXR)/ApoE axis reduce MDSC abundance in murine melanoma models by binding to Lrp8 receptor on MDSC. MDSC blockade by LXR-induced ApoE enhanced cytotoxic T cell activation in B16-F10 bearing mice and patients, eventually leading to reduced melanoma growth. Hence, a further analysis of the innate and adaptive immune responses (including possibly depletion strategies to validate the model) is required to fully understand the implications of apoE in tumor immunity. Another potential limitation of our study is the assumption of cytokine responses observed. Cytokines have complex roles in immune response modulation, and it is also shown that IL-10 is considered to be immune-stimulatory as opposed to immune-suppressive while IL-1a/b can be immune-suppressive. Thus, the results described for ApoE secreted from B16 melanoma tumor cells may impair activation of pro-inflammatory DCs and can be interpreted in an opposite direction. Our results also add to a conflicted literature in which apoE can mediate either pro- or anti-tumor effects. We employ CRISPR gene editing to assess the tumor-intrinsic effects of ApoE. Previous studies have indicated that regardless of the gene being targeted, that CRISPR/Cas9 manipulation can yield tumor cells of increased immunogenicity based on expression of Cas9-derived xenoantigens. Thus, reduced growth rate and increased immunogenicity of ApoE^{-/-} B16 melanoma cells in vivo could be more dependent on Cas9 than ApoE-deficiency, however several of our B16-F10 melanoma controls do have CRISPR/Cas9 expression alone that do not seem to alter immunogenicity.

Despite these contradictions, our findings suggest that apoE plays a substantial immunomodulatory role with multiple inhibitory effects on T cell function, inflammatory cytokine response, and activation of dendritic cells. ApoE also protects tumors by suppressing tumor cell immunogenicity. ApoE^{-/-} B16 cells induced robust immunity in whatever circumstance they were tested either as the primary immunogen in vivo or with re-stimulation of splenocytes ex vivo. Together, the multiple experiments presented, establish the potent inhibitory effects of apoE on immune cell function and activation of immune signaling in the melanoma mouse model. These findings present an opportunity to exploit this pathway for enabling tumor immunity and cancer immunotherapy.

To understand the correlation between immunity and APOE expression in patients with melanoma, the raw RNA-seq data from the TCGA melanoma datasets were accessed (measured by RSEM algorithm) (n=462). Although APOE was abundantly present in the tumors, there was no correlation between APOE and PD1 or PD-L1 expression and there was also no association between APOE and cell infiltrates as there was between PD-L1 and T-cell/neutrophil expression. The findings do not take into consideration the serum levels of ApoE in the patients themselves. The observations from the mouse model imply that the absence of tumor ApoE alone is not sufficient to induce immunity in that the serum level of the host also has significant suppressive effects on induced immunity. In the patient analysis, no association with APOE and inflammatory tumors was found as the case for PD1 and PD-L1, nor was any association with patient survival. ApoE seems to be a potent suppressor of immunity and therefore may act independently of the classic well described checkpoints.

In summary, apoE plays a broad role in immune resistance observed in the WT B16 melanoma tumors. ApoE inhibits activation of immune cells, inflammatory signals, and tumor immunogenicity both locally and systemically via cellular secretion and host production. This work shows that tumor immunity can be restored and enhanced by targeting apoE. These findings identify apoE as a novel tumor checkpoint and an obvious target for improving tumor immunity with cancer immunotherapy.

### Example 11: Materials and Methods

### Animals

Female C57BL/6 mice and apoE^{-/-} mice (B6.129P2-Apoe^{tm1Unc/J}) aged 6 weeks were purchased from Jackson Laboratories (Bar Harbor, Maine, United States). Lrp8^{-/-} C57BL/6 breeder mice were generously provided by Dr. Sohail Tavazoie's laboratory from the Rockefeller Universit: Mice were housed five per cages and kept in a temperature-controlled environment (20±2° C., 50±5% relative humidity) with a 12-hour light/dark cycle in an air-conditioned room with free access 1 food and water. The animals were acclimated for 4-5 days prior to tumor challenge. All procedures were approved by the Institutional Animal Care and Use Committee (IACUC) of Children's Nation Hospital, Washington, DC.

### Cells

The murine melanoma B16-F10 cell line (purchased from ATCC® CRL-6475, VA), were cultured in DMEM (Life Technologies, CA) supplemented with 10% heat-inactivated FBS (Sigma-Aldrich) and 100 IU/ml Penicillin, 100 μg/ml Streptomycin (Life Technologies).

Generation of apoE^{-/-} Cell Lines Via CRISPR Genome Editing in B16-F10 Cells

Six guide RNAs (gRNAs) were designed using IDT's gRNA design tool, targeting the mouse apoE gene. gRNAs were synthesized as 2-part crRNA: tracrRNA gRNAs with chemical modifications (Integrated DNA Technologies, Inc., IA) and were functionally screened by next generation sequencing (NGS) for high INDEL (insertion/deletion) frequency and low in-frame INDEL rates. gRNAs were prepared by complexing a 1:1 molar ratio of the crRNA: tracrRNA at a final concentration of 100 μM, heating to 95° C. and slowly cooling to room temperature. Ribonucleoprotein complexes (RNPs) were formed by the addition of purified Alt-R HiFi Cas9 protein (IDT) to each gRNA at a 1.2:1 molar ratio in 1×PBS to a concentration of 5.6 μM. RNP complexes were allowed to form for 10 min at room temperature before electroporation. RNP complexes (5 μL), Alt-R Cas9 Electroporation Enhancer (3 μL), and 350,000 B16-F10 cells (20 μL) resuspended in Buffer SF were mixed and electroporated using the Lonza 96-well Shuttle System (Lonza, Basel, Switzerland) with electroporation protocol 96-DS-150. Final concentrations for RNF and Alt-R Cas9 Electroporation Enhancer were 1 μM and 4 μM, respectively. Genomic DNA was extracted 48 hours post-transfection using QuickExtract DNA extraction solution (Epicentre Biotechnologies, CA) according to the manufacturer's specifications. The targeting sequence for each ApoE crRNAs are listed in Table 1.

TABLE 1
A list of names and targeting sequences of
crRNAs used in screening for Mm apoE
knock out.

| | | SEQ ID |
|---|---|---|
| crRNA name | Sequence | NO: |
| Mm.Cas9.APOE.1-A | GAGGATCTACGCAACCGACT | SEQ ID |
| | | NO: 1 |
| Mm.Cas9.APOE.1-B | CAACGAGGTGCACACCATGC | SEQ ID |
| | | NO: 2 |
| Mm.Cas9.APOE.1-C | GAGGTGACAGATCAGCTCGA | SEQ ID |
| | | NO: 3 |
| Mm.Cas9.APOE.1-D | GACGCTGTCTGACCAGGTCC | SEQ ID |
| | | NO: 4 |
| Mm.Cas9.APOE.1-E | GGACACTATGACGGAAGTAA | SEQ ID |
| | | NO: 5 |
| Mm.Cas9.APOE.1-F | CTGGTGGAGCAAGGTCGCCA | SEQ ID |
| | | NO: 6 |
| Mm.Cas9.APOE.1-F | CTGGTGGAGCAAGGTCGCCA | SEQ ID |
| | | NO: 7 |

Isolation of Monoclonal apoE Knockouts

The lead gRNA (Mm. Cas9.APOE.1-E) resulted in a 99% INDEL frequency with no in-frame INDELs and was electroporated into B16-F10 cells using the Lonza 96-well Shuttle System a previously described. The electroporated cells were plated in 1 well of a 6-well plate and allowed to grow until confluent. The cells were then dissociated by trypsinization, resuspended in media, and counted. The suspension was diluted to 20,000 cells/mL; 4000 cells were added to 1 well of a 96-well plate and then diluted by array dilution. After 5 days of growth, each well was visually screened for single colonies. Wells with only 1 colony were allowed to grow to confluency. Each well was progressively passaged to a larger well until confluent in a 100 mm dish, about 8.8×10\hat{ }6 cells for genomic DNA extraction and further cell passaging. The genomic DNA from each well was subject to quantification of total editing and analysis of INDEL profile by NGS to confirm a monoclonal isolate.

Quantification of total editing and analysis of INDEL profiles by rhAmpSeq

Genomic DNA libraries for sequencing were prepared using IDT rhAmpSeq targeted amplification. In short, the first round of PCR was performed using target-specific primers with universal 5′ tails (Table 2); a second round of PCR incorporated P5 and P7 Illumina adapter sequences to the amplicon ends. Libraries were purified using Agencourt® AMPure® XP system (Beckman Coulter, Brea, CA, USA) with a 1:1 ratio of beads to reaction by volume and quantified with quantitative real-time PCR (qPCR) before loading onto the Illumina® MiSeq platform (Illumina, San Diego, CA, USA). Paired end 150 base pair reads were sequenced using V2 chemistry. A sequencing depth of at least 1000 reads was obtained for each sample. Total editing efficiency was calculated and INDEL profiles were evaluated using an IDT custom-built pipeline, CRISPAltRations.

TABLE 2

| Primer | | SEQ ID |
|---|---|---|
| name | Sequence | NO: |
| NGS | acactctttccctacacgacgctct | SEQ ID |
| For 1 | tccgatctAGACCCAAAAAGACTGT | NO: 8 |
| | AGG | |
| NGS | gtgactggagttcagacgtgtgctc | SEQ ID |
| Rev 1 | ttccgatctTGCCGAGGGTGAAAGA | NO: 9 |
| | GCTG | |
| NGS | acactctttccctacacgacgctct | SEQ ID |
| For 2 | tccgatctGCCTTCATCTCCTTCCT | NO: 10 |
| | GTG | |
| NGS | gtgactggagttcagacgtgtgctc | SEQ ID |
| Rev 2 | ttccgatctCCTCTGTGCTCTGGCC | NO: 11 |
| | CAGC | |
| NGS | acactctttccctacacgacgctct | SEQ ID |
| For 3 | tccgatctAGGCTGGGCAAAGAGGT | NO: 12 |
| | GCA | |
| NGS | gtgactggagttcagacgtgtgctc | SEQ ID |
| Rev 3 | ttccgatctCGCTTCTGCAGATCCT | NO: 13 |
| | CGGC | |
| NGS | acactctttccctacacgacgctct | SEQ ID |
| For 4 | tccgatctTGCCGAGGATCTGCAGA | NO: 14 |
| | AGC | |
| NGS | gtgactggagttcagacgtgtgctc | SEQ ID |
| Rev 4 | ttccgatctGCCGCCCTCGGATGCG | NO: 15 |
| | GTCA | |

### Antibodies and Reagents

Anti (α)-mouse CTLA-4, and mouse IgG2b isotype antibodies were purchased from BioXCell (West Lebanon, NH). COG133 and JQ1 were purchased from Tocris (Minneapolis, MN). Dynabeads™ Mouse T-Activator CD3/CD28 for T-Cell Expansion and Activation kit, Vybrant™ DyeCycle™ Violet Stain kit, SYTOX™ red dead cell stain kit, CellTrace™ far red cell proliferation kit, Live/Dead fixable aqua dead cell stain kit, Brilliant stain buffer and mouse IL-2 Carrier-Free recombinant protein were purchased from Thermo Fisher (Waltham, MA).

### Multiplex Cytokine/Chemokine Analysis

Cell culture supernatant was collected after centrifuge at 1,200 rpm for 10 min at 4° C. The concentrations of the following immune molecules were determined using the mouse Cytokine & Chemokine 36-plex ProcartaPlex Panel, a magnetic bead-based multiplex immunoassay (Thermo Scientific) following our previous protocol(19). Briefly, cell culture supernatant samples were mixed with antibody-linked polystyrene beads on a 96-well plate and incubated at room temperature (RT) for 2 h on an orbital shaker at 500 rpm. After washing, plates were incubated with biotinylated detection antibody for 30 min at RT. Plates were then washed twice and then labeled beads were resuspended in streptavidin-PE. Each sample was measured in duplicate along with standards (8-point dilutions) and the buffer control. Plates were read using a Luminex Bio-plex 200 system (Bio-Rad Corp.) for quantitative analysis.

Isolating T Cells from Mouse Spleen

Spleens were collected from mice euthanized by C02 narcosis and cervical dislocation. Spleens were pulverized through a 40-μm mesh cell strainer and treated with ACK lysing buffer (Thermo Fisher) for 10 seconds to remove erythrocytes. According to the manufacture's instruction of the Pan T Cell Isolation Kit (Miltenyi Biotec), 10 μL of Pan T Cell Biotin Antibody Cocktail was added per 10⁷ splenocytes and incubated for 5 min at 4° C. Subsequently, 20 μL of Pan T Cell MicroBead Cocktail was added per 10⁷ cells. Following incubation for 10 min at 4° C., the mixed cell suspension was applied onto the LS column (Miltenyi Biotec). The flow-through containing unlabeled cells, representing the enriched T cells were collected. T cells were cultured in RPMI 164 media containing 30 U/mL IL-2 (Thermo Fisher) and stimulated with Dynabeads® Mouse T-Activator CD3/CD28 magnetic beads (Thermo Fisher) at a 1:1 ratio (cell:bead).

### IFNγ Measurement

WT and apoE^{-/-} mice or WT and lrp8^{-/-} mice, were inoculated with either WT B16 or apoE^{-/-} B16 cells, with anti-CTLA4 antibody on day 0. These vaccinated splenocytes (VS) were harvested on day 7 and co-cultured with either WT B16 or apoE^{-/-} B16 cells for 48 hr, following which IFNγ levels in media were compared with ELISA assay. To set up co-culture, a total of 5×10⁵ freshly isolated mouse T cells or splenocytes were plated in a volume of 600 μl per well of 24-well plates, then they were co-cultured with 5×10⁴ B16 cells and stimulated with or without CD3/CD28 Dynabeads. Cells were exposed to apoE agonist COG133 at 0, 0.3, 3, 9, 15, 30 μM or human anti-APOE antibody at 1 μg/ml, 10 μg/ml and 20 μg/ml at 37° C. under 5% C02 for 24 hr or 48 hr. Supernatants were collected from triplicate wells, and IFNγ was assayed using the mouse uncoated IFNγ ELISA kit from Invitrogen (Carlsbad, CA). Readings were measured at 450 nm using the EnSpire 2300 Multilabel plate reader (Perkin Elmer, Waltham, Massachusetts, US).

### Cell Lines and Conditioned Media

5×10⁶ wild type (WT) and apoE^{-/-} B16 cells were irradiated at 60 Gy and then cultured in 20 mL DMEM media supplemented with 10% FBS for 48 hr in T75 flask. The conditioned media (CM was collected and centrifuged at 1100 rpm at room temperature for 5 min. The supernatant was aliquoted and stored at −80° C.

### Cell Cycle Assay

For testing cell cycle, 1×10⁶ T cells in 1 mL DMEM complete media were incubated with 5 μM Vybrant™ DyeCycle™ Violet Stain (Thermo Fisher) at 37° C. for 30 min. And then 5 μl 7AAD were added and incubated for 10 min prior to analysis. Total 25,000 cells were analyzed per measurement. The same forward and side scatter gates were applied to each sample, and within that gate we measured the intensity of vibrant cell cycle dye. Samples were analyzed on a flow cytometer using 405 nm excitation and 440 nm emission. To compare the growth rates of B16 WT and apoE^{-/-} cells, the Click-iT Edu Alexa Fluor 488 flow cytometry assay kit was used in conjunction with the FxCycle Violet stain from Invitrogen (Carlsbad, CA). 50,000 cells were plated per well of a 6 well-plate and cell cycle was analyzed at 48 hr as per the manufacturer's directions.

Measurement of apoE in Serum of Mice

Mouse serum was collected from naïve WT C57/BL6 mice and also from both naïve and tumor-bearing C57/BL6 apoE^{-/-} mice. ApoE levels in mouse sera were quantified using the mouse apoE ELISA^{PRO} kit from Mabtech (Cincinnati, OH) as per the manufacturer's directions.

### Nanostring

RNA was extracted and gene expression was directly measured via counts of corresponding mRNA in each sample using an nCounter murine PanCancer Immune Profiling Panel (NanoString, Seattle, WA, USA). For full details, see our previous publication. Briefly, 100 ng of high-quality total RNA were hybridized with reporter probes, and then biotinylated capture probes at 65° C. for 16-18 hr before being placed into the nCounter Prep station in which samples were affixed to a cartridge. Cartridges were then read by the nCounter Digital Analyzer optical scanner. Further advanced immune-profiling analysis was performed using nSolver 4.0 analysis software with nCounter advanced analysis package (NanoString Technologies) with identified immune cell types. Genes were grouped into 14 immune cell types and 39 immune functions according to the manufacturer's designation.

### Quantitative Real-Time RT-PCR

Quantitative real-time PCR (qPCR) was performed using TaqMan® Gene Expression Master Mix (Life Technologies) in a QuantStudio 7 Flex Real-Time PCR System (Thermo Fisher Scientific Waltham, MA) following the methods that we published previously. Each reaction was performed in triplicate, including no template controls and amplification of a housekeeping gene, GAPDH. Gene-specific assays were Mm01307192_m1 for apoE, Mm00464608_m1 for Lrp1, Mm01328171_m1 for Lrp2, Mm00474030_m1 for Lrp8, Mm01177349_m1 for Ldlr, Mm00443298_m1 for Vldlr, Mm99999915_g1 for Gapdh (Life Technologies, Thermo Fisher). Changes in relative gene expression normalized to GAPDH levels were determined using the ΔΔCt method. Results were averaged and statistically analyzed using t-tests.

### Mouse Melanoma Models

C57BL/6 wild type (WT) mice, C57/BL6 apoE knockout (apoE^{-/-}) mice, and C57/BL6 lrp8 knockout (lrp8^{-/-}) mice were injected subcutaneously in the right flank with 1×10⁴ freshly prepared B16 WT or apoE^{-/-} tumor cells in 100 μl 1×PBS on day 0 and euthanized once the tumor reached 20 mm in any dimension. Tumor growth was recorded every day by measuring the diameter in 2 dimensions using a caliper when appropriate as we have previously published. Briefly, tumor volume was calculated using the following formula: large diameter²×small diameter×0.52. A tumor size of 20 mm in diameter in any dimension was designated as the endpoint, and mice were euthanized at that time. Euthanasia was achieved through cervical dislocation after CO2 narcosis. If a tumor impaired the mobility of an animal, became ulcerated, or appeared infected, or a mouse displayed signs of “sick mouse posture”, the mouse was euthanized. All the procedures are approved by the IACUC at CNMC and are in accordance with the humane care of research animals.

### Characterization of Mouse Tumors by Immunohistochemistry (IHC)

Tumor was fixed in 10% neutral buffered formalin (pH 6.8-7.2; Richard-Allan Scientific, Kalamazoo, Michigan, US) for paraffin embedding and sectioning. Five m tissue sections were cut with a microtome, and sample processing and IHC staining were performed as previously described using rabbit polyclonal to CD45 and CD3 antibodies (1:200. Abcam, Cambridge, Massachusetts, US). Isotype-matched antibodies were used for negative controls.

### Statistical Analysis

Statistical analysis of nanostring gene expression, normalization, clustering, Pathview plots and fold-changes were performed using the Advanced Analysis Module in the nSolver™ Analysis Software version 4.0 from NanoString Technologies (NanoString Technologies, WA, USA) following our published methods(19). Briefly, raw data for each sample were normalized to the geometric mean of housekeeping genes using the geNorm algorithm. Pathway scores were calculate as the first principal component (PC) of the pathway genes' normalized expression. Each cell type score has been centered to have mean 0 and as abundance estimates (cell type scores) are calculated in log 2 scale, an increase of 1 corresponds to a doubling in abundance. All differentially expressed genes were subjected to KEGG term analysis, with significance accepted at p<0.05. The Benjamin: Yekutieli method was used to control the false discovery rate. All statistical analyses of nanostring data were carried out in R v3.4.3 software.

Statistical significance for each set of experiments was determined by the unpaired 2-tailed Student's t-test, and the specific tests were indicated in the figure legends. The data are expressed a, the mean (±SD), with p<0.05 considered statistically significant.

### Human Melanoma RNA-Seq Analysis

The raw RNA-seq data of melanoma biopsies were accessed through the Gene Expression Omnibus database (accession number: GSE78220)(21)—Raw reads mapping to the reference genome (GRCh38) were performed on quality-checked and trimmed reads using STAR 2.4.1c (22). The reference annotation is Ensembl v86. The overlap of reads with annotation features found in the reference.gtf were calculated using HT-seq v0.6.1(23). The output computed for each sample (raw read counts) was then used as input for DESeq2(24) analysis. Raw counts were normalized using DESeq2 function “rlog,” and normalized counts were used for downstream analysis. Statistical calculations were performed using GraphPad Prism software (GraphPad Software, San Diego, CA, USA) or R Software (Version 4.0).

### REFERENCE

1. Stojakovic T, Scharnagl H, Marz W. ApoE: crossroads between Alzheimer's disease and atherosclerosis. Semin Vase Med. 2004; 4(3):279-85.
2. Bonacina F, Coe D, Wang G, Longhi M P, Baragetti A, Moregola A, et al. Myeloid apolipoprotein E controls dendritic cell antigen presentation and T cell activation. Nat Commun. 2018; 9(1):3083.
3. Wang X, Huang R, Zhang L, Li S, Luo J, Gu Y, et al. A severe atherosclerosis mouse model on the resistant NOD background. Dis Model Mech. 2018; 11(10).
4. Yin C, Ackermann S, Ma Z, Mohanta S K, Zhang C, Li Y, et al. ApoE attenuates unresolvable inflammation by complex formation with activated C1q. Nat Med. 2019; 25(3):496-506.
5. Ostendorf B N, Bilanovic J, Adaku N, Tafreshian K N, Tavora B, Vaughan R D, et al. Common germline variants of the human APOE gene modulate melanoma progression and survival. Nat Med. 2020; 26(7):1048-53.
6. Lee Y S, Yeo I J, Kim K C, Han S B, Hong J T. Inhibition of Lung Tumor Development in ApoE Knockout Mice via Enhancement of TREM-1 Dependent NK Cell Cytotoxicity. Front Immunol. 2019; 10:1379.
7. An H J, Koh H M, Song D H. Apolipoprotein E is a predictive marker for assessing non-small cell lung cancer patients with lymph node metastasis. Pathol Res Pract. 2019; 215(10):152607.
8. Zhao Z, Zou S, Guan X, Wang M, Jiang Z, Liu Z, et al. Apolipoprotein E Overexpression Is Associated With Tumor Progression and Poor Survival in Colorectal Cancer. Front Genet. 2018; 9:650.
9. Fang Z, He M, Song M. Serum lipid profiles and risk of colorectal cancer: a prospective cohort study in the UK Biobank. Br J Cancer. 2021; 124(3):663-70.
10. Nicoll J A, Zunarelli E, Rampling R, Murray L S, Papanastassiou V, Stewart J. Involvement of apolipoprotein E in glioblastoma: immunohistochemistry and clinical outcome. Neuroreport. 2003; 14(15):1923-6.
11. Venanzoni M C, Giunta S, Muraro G B, Storari L, Crescini C, Mazzucchelli R, et al. Apolipoprotein E expression in localized prostate cancers. Int J Oncol. 2003; 22(4):779-86.
12. Su W P, Chen Y T, Lai W W, Lin C C, Yan J J, Su W C. Apolipoprotein E expression promotes lung adenocarcinoma proliferation and migration and as a potential survival marker in lung cancer. Lung Cancer. 2011; 71(1):28-33.
13. Huvila J, Brandt A, Rojas C R, Pasanen S, Talve L, Hirsimaki P, et al. Gene expression profiling of endometrial adenocarcinomas reveals increased apolipoprotein E expression in poorly differentiated tumors. Int J Gynecol Cancer. 2009; 19(7):1226-31.
14. Oue N, Hamai Y, Mitani Y, Matsumura S, Oshimo Y, Aung P P, et al. Gene expression profile of gastric carcinoma: identification of genes and tags potentially involved in invasion, metastasis, and carcinogenesis by serial analysis of gene expression. Cancer Res. 2004; 64(7):2397-405.
15. Kemp S B, Carpenter E S, Steele N G, Donahue K L, Nwosu Z C, Pacheco A, et al. Apolipoprotein E Promotes Immune Suppression in Pancreatic Cancer through NF-kappaB-Mediated Production of CXCL1. Cancer Res. 2021; 81(16):4305-18.
16. Alikhani N, Ferguson R D, Novosyadlyy R, Gallagher E J, Scheinman E J, Yakar S, et al. Mammary tumor growth and pulmonary metastasis are enhanced in a hyperlipidemic mouse model. Oncogene. 2013; 32(8):961-7.
17. Pencheva N, Tran H, Buss C, Huh D, Drobnjak M, Busam K, et al. Convergent multi-miRNA targeting of ApoE drives LRP1/LRP8-dependent melanoma metastasis and angiogenesis. Cell. 2012; 151(5):1068-82.
18. Tavazoie M F, Pollack I, Tanqueco R, Ostendorf B N, Reis B S, Gonsalves F C, et al. LXR/ApoE Activation Restricts Innate Immune Suppression in Cancer. Cell. 2018; 172(4):825-40 e18.
19. Wu X, Nelson M, Basu M, Srinivasan P, Lazarski C, Zhang P, et al. MYC oncogene is associated with suppression of tumor immunity and targeting Myc induces tumor cell immunogenicity for therapeutic whole cell vaccination. J Immunother Cancer. 2021; 9(3).
20. Chakrabarti L, Morgan C, Sandler A D. Combination of Id2 Knockdown Whole Tumor Cells and Checkpoint Blockade: A Potent Vaccine Strategy in a Mouse Neuroblastoma Model. PLoS One. 2015; 10(6):e0129237.
21. Hugo W, Zaretsky J M, Sun L, Song C, Moreno B H, Hu-Lieskovan S, et al. Genomic and Transcriptomic Features of Response to Anti-PD-1 Therapy in Metastatic Melanoma. Cell. 2016; 165(1):35-44.
22. Dobin A, Davis C A, Schlesinger F, Drenkow J, Zaleski C, Jha S, et al. STAR: ultrafast universal RNA-seq aligner. Bioinformatics. 2013; 29(1):15-21.
23. Noble M L, Kelley D L. Accuracy of tri-axial cinematographic analysis in determining parameters of curvilinear motion. Res Q. 1969; 40(3):643-5.
24. Love M I, Huber W, Anders S. Moderated estimation of fold change and dispersion for RNA-seq data with DESeq2. Genome Biol. 2014; 15(12):550.
25. Bilousova T, Melnik M, Miyoshi E, Gonzalez B L, Poon W W, Vinters H V, et al. Apolipoprotein E/Amyloid-beta Complex Accumulates in Alzheimer Disease Cortical Synapses via Apolipoprotein E Receptors and Is Enhanced by APOE4. Am J Pathol. 2019; 189(8):1621-36.
26. Herz J. Apolipoprotein E receptors in the nervous system. Curr Opin Lipidol. 2009; 20(3):190-6.
27. Laskowitz D T, Lee D M, Schmechel D, Staats H F. Altered immune responses in apolipoprotein E-deficient mice. J Lipid Res. 2000; 41(4):613-20.
28. Baitsch D, Bock H H, Engel T, Telgmann R, Muller-Tidow C, Varga G, et al. Apolipoprotein E induces antiinflammatory phenotype in macrophages. Arterioscler Thromb Vasc Biol. 2011; 31(5):1160-8.
29. Zhu Y, Kodvawala A, Hui D Y. Apolipoprotein E inhibits toll-like receptor (TLR)-3- and TLR-4-mediated macrophage activation through distinct mechanisms. Biochem J. 2010; 428(1):47-54.
30. Tenger C, Zhou X. Apolipoprotein E modulates immune activation by acting on the antigen-presenting cell. Immunology. 2003; 109(3):392-7.
31. Wang Y, Sun S N, Liu Q, Yu Y Y, Guo J, Wang K, et al. Autocrine Complement Inhibits IL10-Dependent T-cell-Mediated Antitumor Immunity to Promote Tumor Progression. Cancer Discov. 2016; 6(9):1022-35.
32. Mehta A, Merkel O M. Immunogenicity of Cas9 Protein. J Pharm Sci. 2020; 109(1):62-7.

What is claimed is:

1. A method for treating a cancer in a subject, wherein the cancer expresses and/or secrets apolipoprotein E (apoE), the method comprising inhibiting expression or activity of apoE, thereby treating the cancer in the subject.

2. The method of claim 1, comprising administering to the subject a therapeutically effective amount of an agent that inhibits expression or activity of apoE.

3. The method of any one of the preceding claims, wherein the agent that inhibits expression of apoE is selected from the group consisting of an RNAi, an siRNA, an miRNA, an antisense oligo- or polynucleotide, a guide RNA, and a polynucleotide coding sequence thereof on a vector.

4. The method of claim 1, comprising administering to the subject a therapeutically effective amount of an agent that inhibits activity of apoE.

5. The method of claim 4, wherein the agent that inhibits activity of apoE comprises a peptide, protein or a small molecule that binds to apoE or a receptor of apoE (such as lrp1, lrp8 and/or ldlr).

6. The method of claim 4 or 5, wherein the protein that inhibits activity of apoE is an anti-apoE or apoE receptor antagonist/neutralizing antibody or an antigen-binding fragment thereof.

7. The method of claim 6, wherein the anti-apoE or anti-apoE receptor antibody or an antigen-binding fragment thereof is a monoclonal antibody, a humanized antibody, an Fab, Fab′, F(ab′)₂, Fd, single chain Fv or scFv, disulfide linked Fv, V-NAR domain, IgNar, intrabody, IgGΔCH2, minibody, F(ab′)₃, tetrabody, triabody, diabody, single-domain antibody, DVD-Ig, Fcab, mAb₂, (scFv)₂, or scFv-Fc.

8. The method of any one of the preceding claims, wherein the cancer is a solid tumor.

9. The method of any one of the preceding claims, wherein the cancer is selected from esophageal carcinoma, lung squamous cell carcinoma, lung adenocarcinoma, cervical squamous cell carcinoma, endometrial adenocarcinoma, bladder cancer, urothelial carcinoma, lung cancer, non-small cell lung cancer, colorectal cancer, rectal cancer, endometrial cancer, skin cancer, head & neck squamous cell carcinoma, brain cancer, glioblastoma multiforme, non-CNS tumor, breast cancer, gastric cancer, gastroesophageal cancer, gastroesophageal adenocarcinoma, pancreatic cancer, prostate cancer, ovarian cancer, melanoma, nasopharyngeal carcinoma, anal carcinoma, mesothelioma, renal cell carcinoma, gallbladder/cholangiocarcinoma, pancreatic carcinoma, penile squamous cell carcinoma, and vulvovaginal carcinoma.

10. The method of any one of the preceding claims, wherein the cancer is melanoma.

11. The method of any one of the preceding claims, wherein the subject is human.

12. The method of any one of the preceding claims, wherein the subject is administered a second anti-cancer therapy.

13. The method of any one of claim 12, wherein the second anti-cancer therapy is selected from the group consisting of surgery, radiation, adoptive cell therapy (e.g., CAR T cell therapy), a cancer vaccine, a chemotherapeutic agent, an anti-angiogenesis agent, a growth inhibitory agent, an immunotherapy, and/or an anti-neoplastic composition.

14. The method of claim 12 or 13, wherein the second anti-cancer therapy comprises administering an immune checkpoint inhibitor.

15. The method of claim 14, wherein the immune checkpoint inhibitor is an anti-CTLA4, anti-PD1 or anti-PDL1 inhibitor (e.g., antibody or antigen-binding fragment thereof).

16. The method of any one of claims 2-15, wherein the agent is administered intravenously, subcutaneously, or intratumorally.
